(19) United States
(12) Patent Application Publication
(10) Pub. No.: US 2023/0277115 A1
(43) Pub. Date: Sep. 7, 2023

(54) DIAGNOSIS AND TREATMENT OF PELVIC CONDITIONS

(71) Applicant: NATIONAL UNIVERSITY OF IRELAND, GALWAY, Galway (IE)

(72) Inventors: Sinead HUGHES, Batterstown (IE); Siobhan KELLEHER, Salthill (IE)

(73) Assignee: NATIONAL UNIVERSITY OF IRELAND, GALWAY, Galway (IE)

(21) Appl. No.: 18/016,973

(22) PCT Filed: Jul. 20, 2021
(86) PCT No.: PCT/EP2021/070307

§ 371 Date: Jan. 19, 2023

(30) Foreign Application Priority Data
Jul. 20, 2020 (EP) 20186792.6

(51) Int. Cl. A61B 5/391; A61B 5/00; G16H 50/70; G16H 50/30

(52) U.S. Cl. CPC A61B 5/391; A61B 5/7264; G16H 50/70; G16H 50/30

(57) ABSTRACT

A system to determine status of a pelvic condition in a subject characterised by abnormal contractility activity of a target pelvic structure is described. The system comprises a sensing module to measure electrical activity of the subjects pelvis at a plurality of time points during the subjects hormonal cycle, a signal processing module configured to receive electrical activity measurements from the sensing module and isolate from the electrical activity measurements electrical contractility parameter measurements representative of the target pelvic structure, and a processor module operably connected to the signal processing module. The processor is configured to receive as an input the electrical contractility parameter measurements representative of the target pelvic structure, generate a data profile of the subject comprising the electrical contractility parameter measurements representative of the target pelvic structure, compare the data profile with a database of reference data profiles comprising reference data profiles of subjects with different pelvic condition status, output the status of the pelvic condition in the subject based on the comparison. In any embodiment, the signal processing module is configured to isolate from the electrical activity measurements slow wave electrical contractility parameter measurements representative of the target pelvic structure. Systems and methods for treating pelvic conditions comprising stimulation of a pelvic structure to normalise pelvic structure contractility are also described.

### FIELD OF THE INVENTION

The present invention relates to method and device for diagnosis of a pelvic condition such as endometriosis, prostatitis or benign prostatic hyperplasia. The invention also relates to a method and device for treating a pelvic condition.

### BACKGROUND TO THE INVENTION

Endocrine hormones (e.g., cortisol, thyroid hormone, sex steroids, GH) are regulated by complex reciprocal interactions among the hypothalamus, anterior pituitary, and adrenal glands—the hypothalamic-pituitary-adrenal axis. This central control mechanism is responsible for circulating gonadal sex steroid hormones after puberty, estrogens in females and testosterone in males. Disturbance of this mechanism may occur as a result of either an environmental change (stress, estrogen-like pollutants, endocrine-disrupting compounds in diet), ageing, or as a result of disease, either directly affecting the central hypothalamic-pituitary-adrenal axis or altering the local hormonal milieu in tissues. The loss of hormonal balance results in diseases for example depression and inflammatory disorders. Tissues where injury, inflammation and motility are influenced by sex steroid hormones, for example estrogen, could include the brain, endocrine glands, endocrine system, immune system, lungs, cardiovascular system, genitor-urinary, reproductive system.

The process of maintaining a suitable environment for pelvic function is a complex one which involves local and central control mechanisms and the interplay of the endocrine and immune system. Although the roles of estrogens in gonadal organs are well understood, many studies have highlighted a role for localized estrogen production in modulation of smooth muscle tone of visceral organs of the pelvic cavity, with or without dependency on circulating estrogen. In females, with conditions such as endometriosis and adenomyosis the concentration of estradiol in menstrual blood is higher than healthy women, whereas as the respective peripheral levels were the same (Takahashi et al. 1989). In males, conditions such as benign prostate hyperplasia are linked with increased serum estrogen level and increased urinary estrogen content (Sodani 2018). Therefore, autocrine and paracrine function underly these pelvic conditions and are at least partly regulated by sex steroids. Reciprocal interactions of cytokines and other components of the immune system interact with the endocrine system. These interactions of these two systems are responsible for many pelvic conditions in men and women. Inflammation is the basic process whereby tissues of the body respond to injury. Men and women have different hormonal exposures, potentially contributing to different injury rates. Furthermore, different phases of men's or women's life can change the injury risk level to pelvic organs and structures owing to different hormonal milieus (Bowmin-Colin et al. 2016).

Patterns of sex-steroid exposure varies over the day and life for both sexes, and in addition, cyclically for females during their reproductive years. After puberty, the rise in gonadal steroids in males and females activates reproductive organs in the pelvic cavity. The female ovaries and uterus are exposed to a cyclical pattern of the main gonadal steroid, estradiol for a certain period of adult life, until levels fall precipitously at reproductive senescence or menopause. In contrast, male testes and prostate is exposed to a relatively steady level of the main gonadal steroid, testosterone, for most of adult life. However, as men age, the amount of active testosterone in their blood decreases, which leaves a higher proportion of estrogen.

These gonadal-derived hormones are released into general circulation and target distal hormone-responsive visceral organs in the pelvic cavity. This greater estrogen dominance in aging males increases smooth muscle tone in the prostate. In females, the cyclical pattern over the reproductive years has a more complex effect on visceral organs especially the uterus. The amplitude, frequency, basal tone and direction of uterine contractions (UC) correlates with different stages of the hormonal cycle.

However, injury to visceral organs can lead to increased inflammation and contractility resulting in pathological pelvic conditions. For example, abnormal uterine contractility is associated with endometriosis (Bulletti et al. 1997), (Kido et al. 2007), polycystic ovary syndrome (Sajadi et al. 2018), endometritis (Pinto et al. 2015), uterine leiomyoma (Kido et al. 2014), and ovarian cancer (Modzelewska et al. 2017) and may also underlie other common and important disorders such as infertility, implantation failure, dysmenorrhea, spontaneous miscarriage, or preterm birth (Aguilar et al. 2010).

In men, prostate smooth muscle contractility plays a role in the pathophysiology of pelvic conditions such as lower urinary tract symptoms (LUTS) (Hennenberg et al 2018), Benign Prostatic Hyperplasia (BPH) (Kugler et al 2017) and prostatitis.

However, heightened contractility of one organ, for example the uterus, can contribute to changes in in tone of other pelvic structures (this region of the body contains the uterus, ovaries, cervix, vagina and the clitoris along with the 5 pelvic bones, muscles, ligaments, nerves, blood vessels, bladder, urethra, colon and rectum) due to paracrine changes such as the altered hormonal and inflammatory milieu. In the example of endometriosis, where uterine contractility is elevated, this manifests as an inflammatory disorder of the pelvic viscera which elicits noxious stimuli to the sacral cord that sets up a pelvic floor muscle dysfunction with sacral nerve hypersensitivity and a sacral cord wind-up. The guarding reflex is a viscero-muscular reflex activated with the aim of increasing the tone of the pelvic floor during routine daytime activity. In these patients, there is an afferent autonomic bombardment that can enhance and maintain a guarding reflex that manifests itself as a hypertonia of the pelvic floor. Other pain disorders, such as irritable bowel syndrome, inflammatory bowel disease, interstitial cystitis, fibromyalgia, and vulvodynia are all found to have a pelvic hypertonia. Frequently, chronic pelvic pain (CPP) is characterized by an overlapping of these different conditions. Similarly in men, prostatic inflammation influences other pelvic structures such as bladder sensation and function.

Altered contractility of any of the pelvic organs or structures, whether caused directly by an injury or indirectly from cross talk from another organ contributes to many pelvic conditions including; endometriosis, adenomyosis, endometritis, chronic pelvic pain, benign prostate hyperplasia, prostatitis, interstitial cystitis, pelvic inflammatory disease, irritable bowel syndrome, inflammatory bowel disease, heavy menstrual bleeding, dysfunctional uterine bleeding, hormone-dependent cancers of the pelvic (ovarian, uterine, endometrial, prostate, testicular, bladder), polycystic ovary syndrome, follicular maturation arrest, anovulation, dysmenorrhea, anovulation, infertility, uterine leiomyoma, precocious puberty, endometritis, erectile dysfunction, incontinence (faecal incontinence, stress urinary incontinence, urge incontinence, mixed incontinence), pelvic floor myalgia, pelvic floor dysfunction, interstitial cystitis, dysuria (painful urination), dyspareunia (pain during intercourse), dyschezia (painful defaecation), dysorgasmia (painful ejaculation). WO2019/016759 describes a system for uterine activity monitoring in a pregnant woman involving monitoring electrical activity of the uterus, extracting uterine electrical activity characteristics, and analysing the electrical activity characteristics to classify the uterine activity as one of several labor conditions including pre-term labor contraction and labor contractions. Uterine contractility associated with pregnant women are generally measured in the 0.3 to 5 Hz frequency range.

It is an object of the invention to overcome at least one of the above-referenced problems.

### SUMMARY OF THE INVENTION

The Applicant has discovered that contractile parameters of a pelvic structure in a non-pregnant subject mapped over a time period such as a hormonal cycle (e.g. the menstrual cycle in a non-pregnant female), or specific stages of the hormonal cycle, differ between subjects with a pelvic conditions and subjects that are free of the pelvic condition, and can therefore be used to determine the status of a pelvic condition in the subject. The Applicant has also discovered that contractile parameters can be measured non-invasively using a wearable sensor, allowing the measurement of contractile parameters over an extended period of time. In a specific aspect, the system and methods of the invention isolate slow waves characteristic of a target pelvic organ and employ the slow wave signal or a feature extracted from the slow waves as a diagnostic variable of a pelvic condition. An example of a slow wave signal used in one aspect of the system and methods of the invention is uterine myometrial motility which has a frequency in the 0.00 to 0.05 Hz range. The Applicant demonstrates herein that this slow wave signal can be isolated using an external wearable sensor, processed, and compared with reference signals to identify endocrine conditions such as endometriosis and associated conditions like. In a related aspect, the Applicant has discovered that electrical stimulation of the target structure during specific stages of the hormonal cycle can be used to normalise abnormal contractile activity of a pelvic structure and therefore treat or prevent pelvic disease. For example, in the case of a female subject with endometriosis, the Applicant has discovered that application of electrostimulation therapy specifically during the follicular stage of the subject's hormonal cycle normalises contractile activity of the uterus.

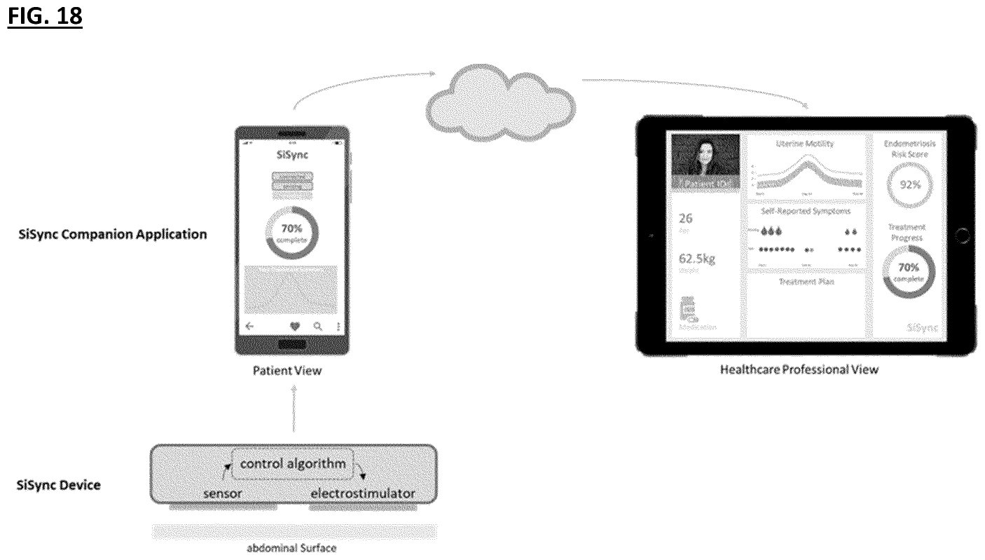

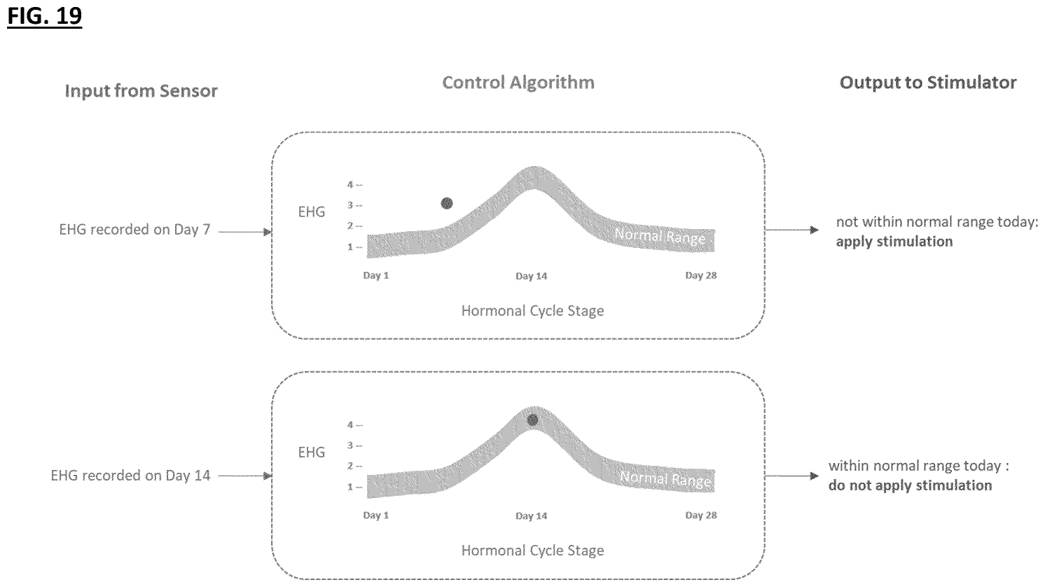

The Applicant therefore provides a system to determine status of a pelvic condition in the subject that employs a non-invasive sensor to measure a contractile parameter of a target pelvic structure (such as the uterus in a female or the prostate in a male) at time points during the hormonal cycle (e.g. the menstrual system in a non-pregnant female) and a connected processor configured to compile the measurements into a data profile, and correlate the data profile with pelvic condition status using, e.g., a computational classification model generated with reference data profiles. The system may in one aspect also include a pelvic structure stimulation model that is non-invasive, and the processor may be configured to actuate the stimulation model upon detection of a pelvic condition. The processor may also be configured to monitor the hormonal cycle in the subject and actuate the stimulation module during a specific stage in the hormonal cycle. In one embodiment, the processor is configured to actuate the stimulation module (typically via a controller) at a stage in the subject's hormonal cycle when abnormal contractile parameter activity is detected by the processor (closed loop system illustrated in FIGS. 18 and 19).

In a first aspect, the invention provides a system to determine status of a pelvic condition in a subject, generally a non-pregnant subject, characterised by abnormal contractility activity of a target pelvic structure, comprising:

a sensing module to measure electrical activity of the subject's pelvis at a plurality of time points during the subject's hormonal cycle;

a signal processing module configured to receive electrical activity measurements from the sensing module and isolate from the electrical activity measurements electrical contractility parameter measurements representative of the target pelvic structure; and a processor module operably connected to the signal processing module and configured to:
receive as an input the electrical contractility parameter measurements representative of the target pelvic structure;
generate a data profile of the subject comprising the electrical contractility parameter measurements representative of the target pelvic structure;
compare the data profile with a database of reference data profiles; and
output the status of the pelvic condition in the subject based on the comparison.

In any embodiment, the signal processing module is configured to isolate from the electrical activity measurements slow wave electrical contractility parameter measurements representative of the target pelvic structure.

In one embodiment, the processor module is configured to receive as an additional input a plurality of measurements of at least one non-electrical hormonal cycle parameter taken at a plurality of time points during the subject's hormonal cycle, wherein the generated data profile comprises the electrical contractility parameter measurements representative of the target pelvic structure and the non-electrical hormonal cycle parameter measurements.

In one embodiment, the signal processing module comprises a filter corresponding to a characteristic frequency of the target pelvic structure. In one embodiment, the signal processing module comprises a filter corresponding to a characteristic frequency range of slow wave motility of the target pelvic structure. Slow wave motility in a target pelvic organ is the motility of the inner smooth muscle layer, for example the sub-endometrial layer of the myometrium in the uterus or myogenic smooth muscle activity in the prostate in males. Thus, the filter may be configured to isolate the slow wave contractility signal of a target pelvic organ. The filter may be configured to isolate slow waves in the frequency range 0.00 to 0.05 Hz.

In any embodiment, the electrical contractility parameter is a signal comprising or consisting of a slow wave contractility frequency.

In any embodiment, the at least one isolated electrical activity measurement comprises an electrical signal measurement of a signal originating from an inner smooth muscle layer of a pelvic organ comprising or consisting of a low frequency content.

In any embodiment, when the pelvic organ is the uterus, the electrical contractility parameter is a signal originating from the sub-endometrial layer of the myometrium.

In any embodiment, the processor is configured to analyse the generated profile and provide an estimate or calculate a prediction value of whether a health (pelvic) condition is likely to develop based on the generated data profile.

In any embodiment, the signal processing module comprises a filter wherein the filter is configured to isolate the one or more electrical contractility parameter measurements corresponding to a characteristic frequency range of slow wave motility of the target pelvic structure.

In one embodiment, the signal processing module is configured to amplify and digitize the signal.

In one embodiment, the signal processing module is configured to transform the signal into the frequency domain, and isolate signal representative of the target pelvic structure from the overall signal (e.g. pelvic EMG signal), typically by dividing the frequency spectrum of the signal into segments corresponding to the characteristic frequency of each pelvic structure.

In one embodiment, the non-electrical hormonal cycle parameter is selected from pain location, pain intensity, pain occurrence, bleeding occurrence, urinary habits (nocturia, urgency, problems starting or ‘stop-start’), onset of erectile dysfunction for prostate. Bloating, and changes to appetite for ovarian cancer (poor appetite, feeling full quickly). In one embodiment, the processor is configured to record the non-electrical parameter against time and compare with a contractility parameter over time.

In one embodiment, the pelvic condition is an endocrine disorder.

In one embodiment, the subject is a female, and typically a non-pregnant female. In one embodiment, the female subject is an adult or a pubescent female from menarche.

In any embodiment, the subject is a female undergoing in-vitro fertilisation treatment. In this context, the systems and methods of the invention may be employed to monitor the effects of ovarian stimulation and to identify optimal timing and uterine receptivity for embryo transfer. To determine an optimal ovarian stimulation protocol the system and methods of the invention are employed to monitor the uterine response to ovarian stimulation medications. Successful embryo implantation requires the proper timing so the embryo is in the uterus during the 8-10 day window of implantation after ovulation and a uterus optimally ready to receive the embryo. The systems and methods of the invention may therefore be employed during IVF treatment to identify a uterus receptive for embryo implantation. In any embodiment, the methods and system may be configured to stimulate the uterus to make it ready to receive the embryo.

In one embodiment, the subject is female (typically a non-pregnant female) and the target pelvic structure is a uterus or pelvic floor, and the pelvic condition is an endocrine disorder such as endometriosis. The hormonal cycle is generally the menstrual cycle.

In any embodiment, the system and method of the invention is to detect Irritable Bowel Syndrome in a subject. In one embodiment, the subject is a non-pregnant female.

In any embodiment, the system and method of the invention is to detect risk of miscarriage, typically early miscarriage, in a pregnant female. Early miscarriage means miscarriage within 13 weeks of gestation. In any embodiment, the system and method comprises taking measurements of uterine contractility prior to conception or during early pregnancy or both. In any embodiment, elevated uterine motility (for example at or around day 14 of the menstrual cycle) correlates with risk of subsequent miscarriage if the subject becomes pregnant. The system and method of the invention may comprise treatment of a subject identified as being at risk of early miscarriage by electrical stimulation of the uterus to normalise uterine contractility, typically at or around day 14 of the subjects menstrual cycle. FIG. 27.

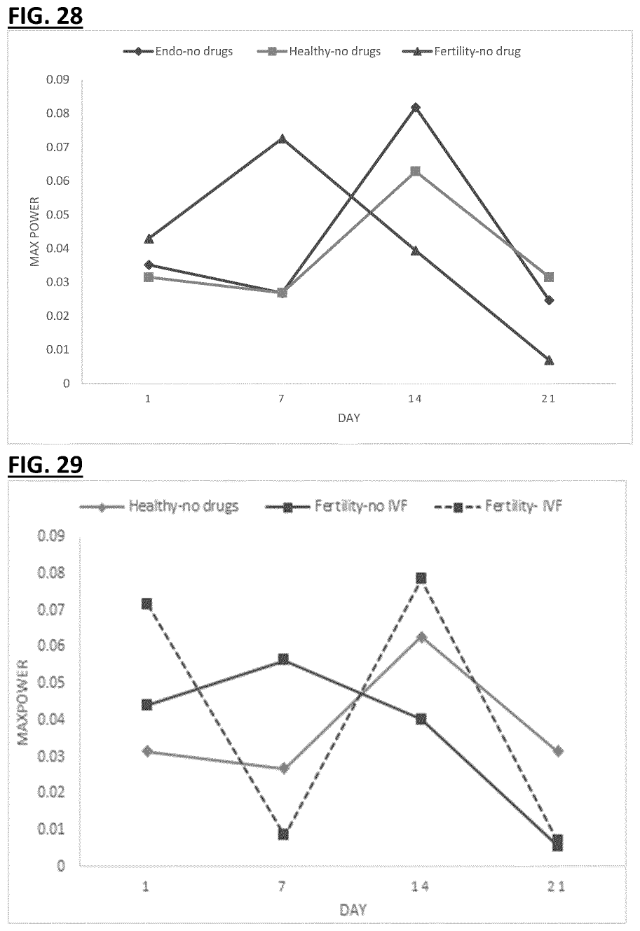

In any embodiment, the system and method of the invention is to detect a female with fertility issues (e.g. infertility or low fertility). In any embodiment, the system and method comprises taking measurements of uterine contractility during the subjects menstrual cycle. In any embodiment, reduced uterine motility at or around day 14 of the menstrual cycle correlates with fertility issues. FIG. 28.

In any embodiment, the system and method of the invention is to ovulation in a non-pregnant female subject. Thus, the invention may be employed to help a woman conceive or to avoid conception.

In any embodiment, the system and method of the invention is to detect an optimal time to harvest eggs from a subject undergoing In-vitro Fertilisation (IVF) therapy. In any embodiment, maximal uterine motility during the cycle correlates with final maturation of eggs and indicates an optimal time for harvesting of eggs during IVF therapy. FIG. 29.

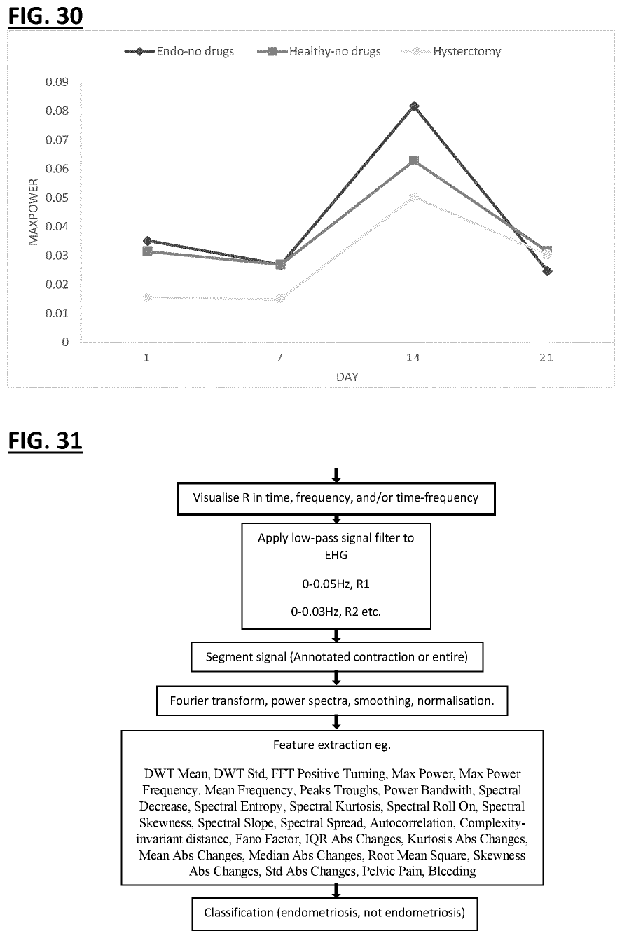

In any embodiment, the system and method of the invention is to monitor a treatment of endometriosis. In any embodiment, the system and method comprises taking measurements of uterine contractility during the treatment period. In any embodiment, a reduction in uterine motility across one or more timepoints during the period of treatment correlates with a reduction in endometriotic lesions and/or treatment effectiveness. FIG. 30.

In any embodiment, the subject is a male.

In any embodiment, the subject is male and the target pelvic structure is a prostate, and the pelvic condition is an endocrine disorder selected from prostatitis, benign prostatic hyperplasia, and prostate cancer. In any embodiment, the at least one isolated electrical activity measurement comprises an electrical signal measurement of a signal originating from myogenic smooth muscle of the prostate comprising or consisting of a low frequency content.

In any embodiment, the processor module is configured to receive as an additional input at least one non-electrical non-hormonal cycle parameter, wherein the generated data profile comprises the electrical contractility parameter measurements representative of the target pelvic structure, the non-electrical non-hormonal cycle parameter measurements, and optionally the non-electrical hormonal cycle parameter measurements.

In any embodiment, the non-electrical non-hormonal cycle parameter is selected from sex, age, reproductive status, hormonal cycle status, previous diagnoses or conditions, family history, medical records, medical imaging, body mass index (BMI), and medication.

In one embodiment, the electrical contractility parameters used for the data profile are extracted from the time domain signal and are selected from frequency, amplitude, intensity and basal tone of target structure contractions.

In one embodiment, the processor is configured to convert a filtered electrical signal to a frequency domain signal using, for example, a fast Fourier Transformation. In one embodiment, the electrical contractility parameters used for the data profile are selected from power spectrum density, DWT Mean, Max Power, and peak frequency. MaxPower means maximum power spectrum density of the signal.

In one embodiment, the electrical contractility parameters are extracted based on independent component analysis.

In one embodiment, the signal processing module is configured to amplify and digitize the electrical signal before extracting the parameter measurements.

In one embodiment, the sensing module is a wearable, non-invasive, sensor.

In one embodiment, the sensor or signal processing module comprises a wireless communications module configured to wirelessly transmit the contractility parameter measurements to the processor, optionally via a communications device.

In one embodiment, the system comprises downloadable software for a mobile communications device configured to cause the mobile communications device to:
receive the contractility parameter measurements from the signal processing module;
communicate the contractility parameter measurements to the processor module;
receive pelvic condition status from the processor module; and
display the received pelvic condition status.

In one embodiment, the downloadable software is configured to allow the subject input the non-electrical hormonal cycle parameter measurements and/or the non-electrical non-hormonal cycle parameter measurements using a user interface of the mobile communications device, and communicate the inputted measurements to the processor module.

In one embodiment, the status of the pelvic condition is selected from positive diagnosis of the pelvic condition, negative diagnosis of the pelvic condition, diagnosis of risk of the pelvic condition developing or occurring, and response of the subject to treatment for the pelvic condition.

In another aspect, the invention provides a system for treating or preventing a pelvic condition in a subject, comprising:
a system for determining pelvic condition status in a subject according to the invention; and
a pelvic structure stimulating module to apply a stimulation treatment to a pelvic structure.

In one embodiment, the pelvic structure stimulating module is non-invasive.

In one embodiment, the pelvic structure stimulating module is wearable.

In one embodiment, the processor is operably connected to the wearable pelvic structure stimulating module and configured to actuate the pelvic structure stimulating module when the status of the pelvic condition in the subject is determined as positive diagnosis of the pelvic condition or risk of development of the pelvic condition.

In one embodiment, the processor is configured to actuate the pelvic structure stimulating module to normalise contractility of the pelvic organ.

In one embodiment, the processor is configured to:
monitor the subject's hormonal cycle using the contractility parameter measurements received from the signal processing module and/or additional subject data obtained at a plurality of time points during the subject's hormonal cycle; and
transiently actuate the pelvic structure stimulating module during a specific stage of the subject's hormonal cycle to, e.g. normalise contractility of the pelvic organ.

In one embodiment, the additional subject data is selected from one or more subject data parameters selected from, temperature, date of last menstruation, and cervical discharge status.

In one embodiment, the processor is configured to actuate the stimulation module (typically via a controller) at a stage in the subject's hormonal cycle when abnormal contractile parameter activity is detected by the processor (closed loop system illustrated in FIGS. 18 and 19).

In one embodiment, the processor is configured to measure the contractility parameter of the target pelvic structure after it has been stimulated, and actuate the pelvic structure stimulating module again if the contractility parameter of the pelvic structure is determined to be abnormal. The processor may be configured to repeat these steps until the contractility parameter sensed by the sensing module is determined to be normalised.

In one embodiment, the sensing module comprises a subject temperature sensor operatively connected to the processor.

In one embodiment, the pelvic structure stimulating module is an electrostimulation module.

In one embodiment, the system comprises a wearable device comprising the sensing module and the wearable pelvic structure stimulating module.

In one embodiment, the wearable device comprises the signal processing module.

In one embodiment, the downloadable software is configured to cause the mobile communications device to:
receive actuating instructions for the pelvic structure stimulating module from the processor module; and
actuate the pelvic structure stimulating module according to the instructions.

In one embodiment, the downloadable software is configured to cause the mobile communications device to display information relating to the actuation of the wearable pelvic structure stimulating module.

In one embodiment, the pelvic condition is endometriosis in which the target pelvic structure is the subject's uterus or an adjacent pelvic structure.

In one embodiment, the pelvic condition is endometriosis in which the target pelvic structure is the subject's uterus or an adjacent pelvic structure, and wherein the processor is configured to actuate the pelvic structure stimulating module during the follicular phase of the subject's hormonal cycle.

In one embodiment, the system comprises a controller configured to control an output parameter of the pelvic structure stimulation module.

In one embodiment, the controller is configured to cause the stimulation module emit electrical pulses of 0.1 to 20 mA.

In one embodiment, the controller is configured to cause the stimulation module to emit electrical pulses with a pulse width of 500 μs to 20 ms.

In one embodiment, the controller is configured to cause the stimulation module emit electrical pulses at a frequency of 0.1 to 50 Hz.

In one embodiment, the controller is configured to actuate the stimulation module for a treatment time of 30-60 minutes.

In one embodiment, the controller is configured to actuate the stimulation module to emit constant current square wave pulses.

In one embodiment, the controller is configured to actuate the stimulation module to emit constant current square wave pulses about at 1-2 mA, about 2 msec/pulse, and with an alternating frequency of about 2/15 Hz.

In another aspect, the invention provides a computer implemented method comprising a processor module operably connected to a signal processing module, said method comprising the steps of:
receiving as an input electrical contractility parameter measurements representative of a target pelvic structure;
generating a data profile of the subject comprising the electrical contractility parameter measurements representative of the target pelvic structure;
comparing the data profile with a database of reference data profiles comprising reference data profiles of subjects with different pelvic condition status; and
outputting a status of the pelvic condition in a particular subject based on the comparison.

In another aspect, the invention provides a method of determining a pelvic condition status in a subject comprising the steps of:
measuring a contractility parameter of a target pelvic structure at a plurality of time points during the hormonal cycle;
preparing a data profile comprising the contractility parameter measurements;
comparing the data profile with one or more reference data profiles; and
determining pelvic condition status based on the comparison.

In any embodiment, the contractility parameter is a slow wave electrical contractility parameter.

In one embodiment, the method includes a step of measuring at least one non-electrical hormonal cycle parameter at a plurality of time points during the subject's hormonal cycle, wherein the data profile comprises the electrical contractility parameter measurements representative of the target pelvic structure and the non-electrical hormonal cycle parameter measurements.

In one embodiment, the pelvic condition is an endocrine disorder.

In any embodiment, the slow wave electrical contractility parameter is frequency, typically contractility frequency in the 0.00 to 0.05 Hz range.

In one embodiment, the target pelvic structure is selected from the uterus, pelvic floor and prostate.

In one embodiment, the subject is female and the target pelvic structure is a uterus or pelvic floor, and the pelvic condition is an endocrine condition such as endometriosis.

In one embodiment, the subject is male and the target pelvic structure is a prostate, and the pelvic condition is a condition of the prostate selected from prostatitis, benign prostatic hyperplasia, and prostate cancer.

In one embodiment, the method includes a step of determining at least one non-electrical non-hormonal cycle parameter, wherein the data profile comprises the electrical contractility parameter measurements representative of the target pelvic structure, the non-electrical non-hormonal cycle parameter measurements, and optionally the non-electrical hormonal cycle parameter measurements.

In one embodiment, the non-electrical non-hormonal cycle parameter is selected from sex, age, reproductive status, hormonal cycle status, previous diagnoses or conditions, family history, medical records, medical imaging, BMI, and medication.

In one embodiment, the electrical contractility parameters are selected from frequency, amplitude, and basal tone of target structure contractions.

In one embodiment, the electrical contractility parameters are measured using a sensing module that is a wearable, non-invasive, sensor.

In another aspect, the invention provides a method of treating a pelvic condition in a subject comprising a step of stimulating a target pelvic structure with a stimulation module.

In one embodiment, the stimulation device is an electrostimulation device.

In one embodiment, the method comprises stimulation of the target pelvic structure with electrical pulses of 0.1 to 20 mA.

In one embodiment, the method comprises stimulation of the target pelvic structure with electrical pulses with a pulse width of 500 μs to 20 ms.

In one embodiment, the method comprises stimulation of the target pelvic organ with electrical pulses at a frequency of 0.1 to 50 Hz.

In one embodiment, the method comprises stimulation of the target pelvic organ for a treatment time of 30-60 minutes.

In one embodiment, the method comprises stimulation of the constant current square wave pulses.

In one embodiment, the method comprises stimulation of the target pelvic structure with constant current square wave pulses about at 1-2 mA, about 2 msec/pulse, and with an alternating frequency of about 2/15 Hz.

In one embodiment the target pelvic structure is stimulated using a non-invasive stimulating module.

In one embodiment, the stimulation is performed during at a specific stage of the hormonal cycle.

In one embodiment, the stimulation is performed during the follicular stage of the hormonal cycle.

In one embodiment, a contractility parameter of the target pelvic structure is determined after stimulation, and a further stimulation treatment is performed if the contractility parameter of the target pelvic structure remains abnormal. These steps may be repeated until the contractility parameter of the target pelvic structure is determined to ne normalised.

In one embodiment, the subject is a female of reproductive age with an endocrine condition (such as endometriosis).

In one embodiment, the subject is a male with a prostate condition such as prostatitis, prostate cancer or benign prostatic hyperplasia.

In one embodiment, the stimulation of the target pelvic structure is configured to normalise abnormal pelvic structure contractile activity.

In another aspect, the invention provides a method of treating endometriosis in a subject comprising a step of administering electrostimulation therapy to the subject's uterus during the follicular phase of the subject's hormonal cycle and not during the ovulatory stage.

In another aspect, the invention provides a wearable device comprising:
a sensing module to measure electrical activity of the subject's pelvis at a plurality of time points during the subject's hormonal cycle;
a signal processing module configured to receive electrical activity measurements from the sensing module and isolate from the electrical activity measurements electrical contractility parameter measurements representative of the target pelvic structure;
a pelvic structure stimulating module to apply a stimulation treatment to a pelvic structure; and optionally, a controller configured to actuate the output parameters of the pelvic structure stimulating module in a pattern configured to normalise the electrical contractility parameter of the target pelvic structure.
In any embodiment, the signal processing module is configured to isolate a slow wave electrical contractility parameter from the electrical activity measurements.

The system may be an electrical medical system. The system may include a real-time operating system. The system may include an embedded platform for automation. The system may include firmware software components. The system may also include an application specific integrated circuit (ASIC), a Programmable Logic Device (PLD) which may include digital circuits, a digital signal processor, a microcontroller or a microprocessor, a memory component and a controller circuit.

The system may include analog interfaces (digital-to-analog, analog-to-digital). The system may include voltage or current regulators and power management circuits. The system may additionally include timing sources.

Other aspects and preferred embodiments of the invention are defined and described in the other claims set out below.

### BRIEF 10

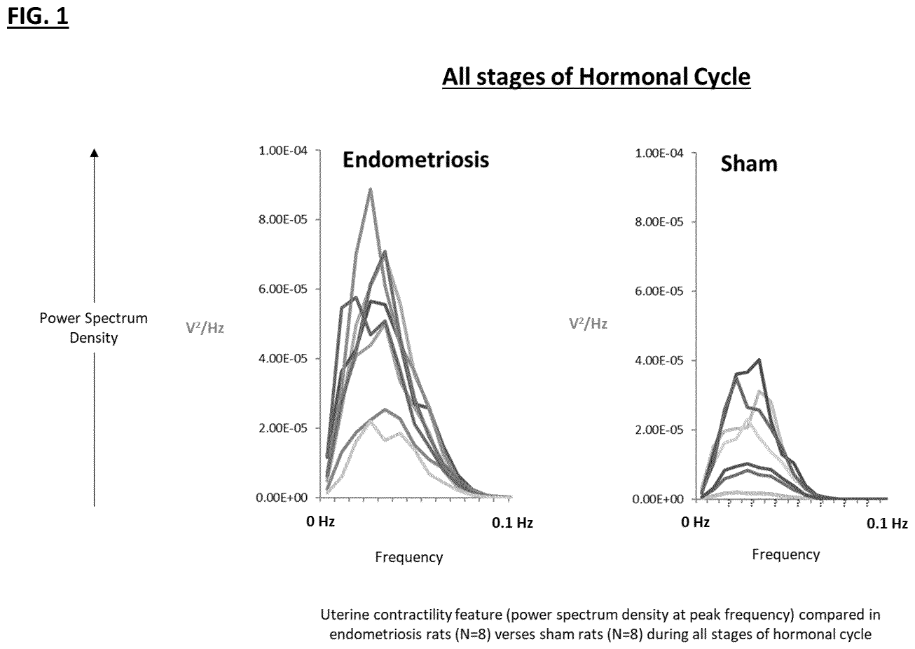

FIG. 1 shows uterine contractility in rats with endometriosis (n=8) and rats without endometriosis (n=8) during all stages of the rats hormonal cycle. Uterine contractility was measured using an electrical sensor and is presented as an electrohysterogram (EHG) transformed into the frequency domain using fast Fourier transform (FFT).

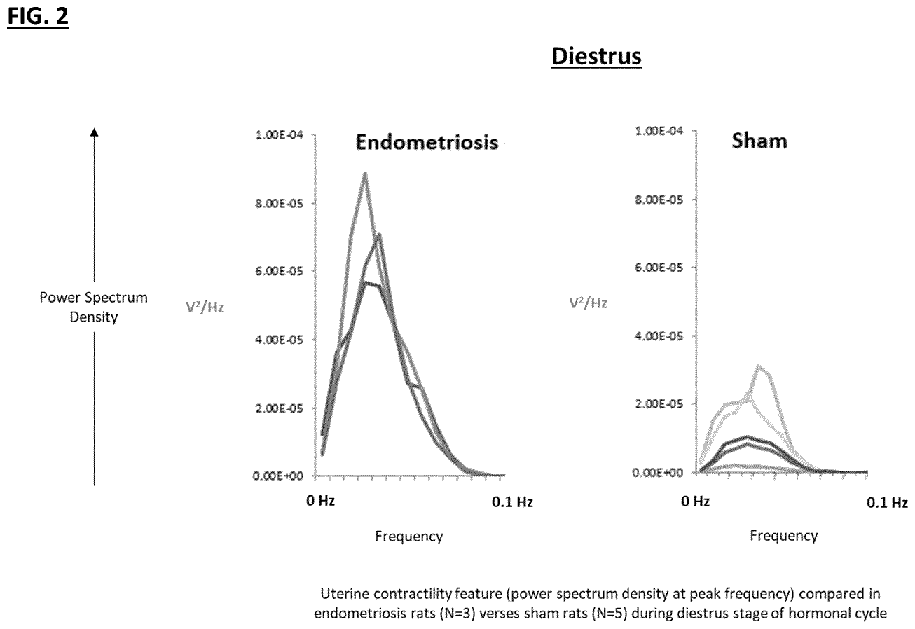

FIG. 2 shows uterine contractility in rats with endometriosis (n=3) and rats without endometriosis (n=5) during the Diestrus stage of the rats hormonal cycle. Uterine contractility was measured using an electrical sensor and is an electrohysterogram (EHG) transformed into the frequency domain using fast Fourier transform (FFT).

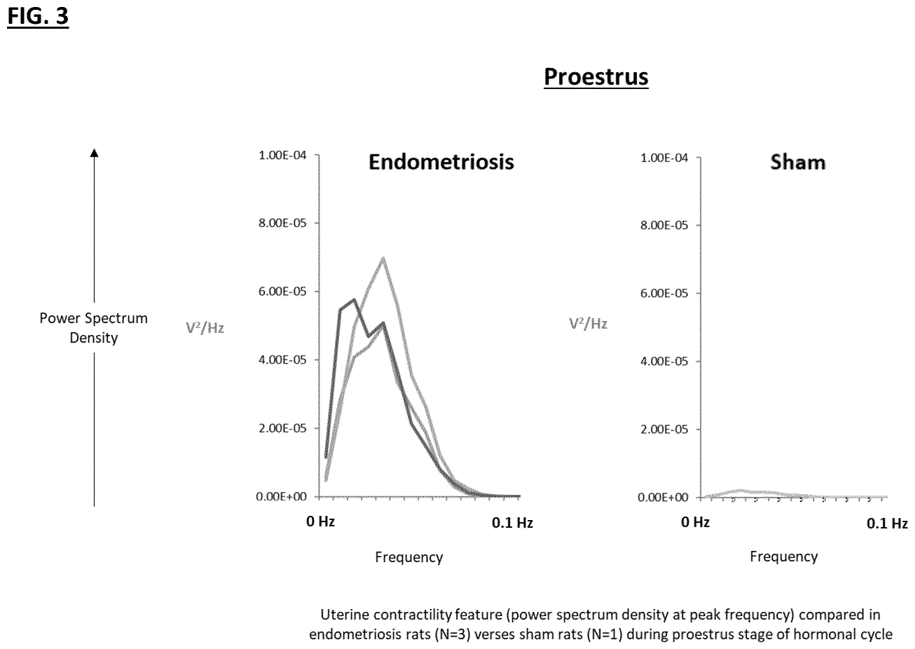

FIG. 3 shows uterine contractility in rats with endometriosis (n=3) and rats without endometriosis (n=1) during the Proestrus stage of the rats hormonal cycle. Uterine contractility was measured using an electrical sensor and is an electrohysterogram (EHG) transformed into the frequency domain using fast Fourier transform (FFT).

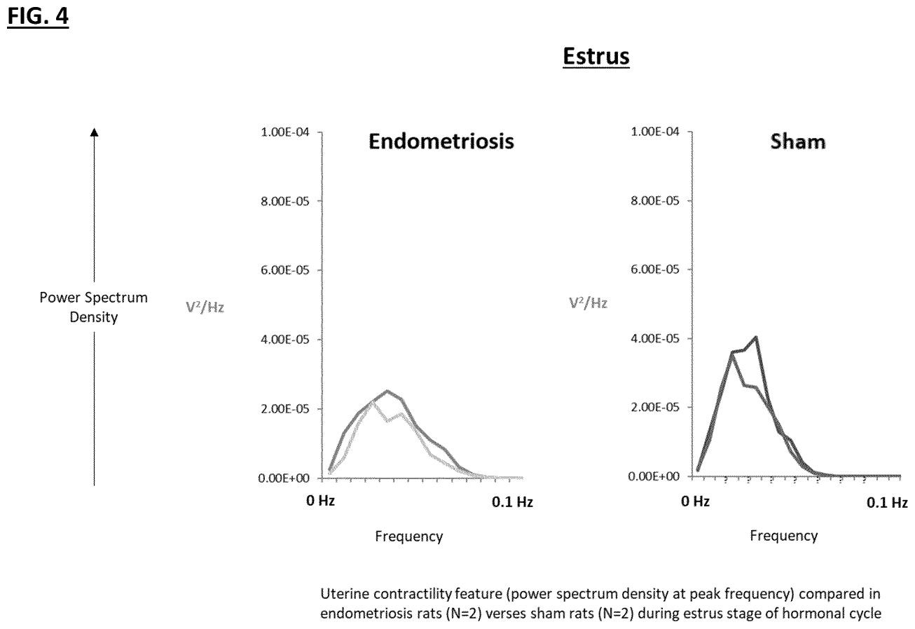

FIG. 4 shows uterine contractility in rats with endometriosis (n=2) and rats without endometriosis (n=2) during the Estrus stage of the rats hormonal cycle. Uterine contractility was measured using an electrical sensor and is presented as an electrohysterogram (EHG) transformed into the frequency domain using fast Fourier transform (FFT).

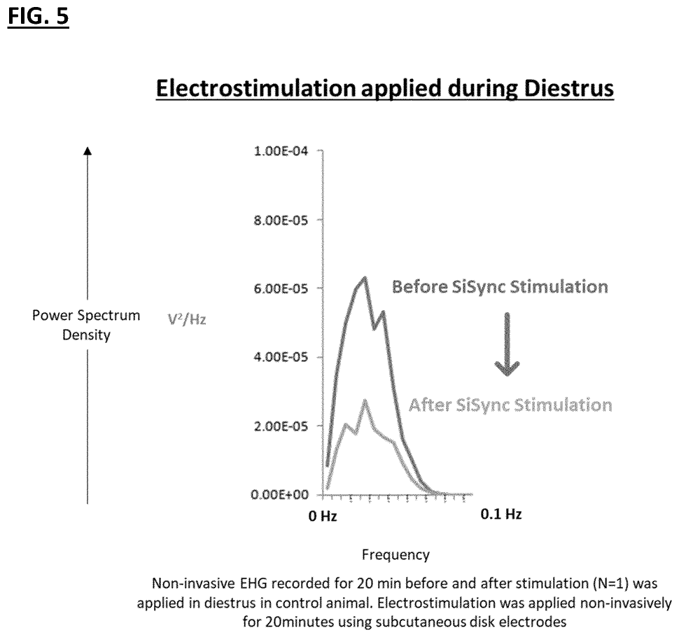

FIG. 5 shows that uterine contractility in rats can be reduced by electrostimulation of the uterus using a non-invasive electrostimulation electrode. Uterine contractility was measured using an electrical sensor and is presented as an electrohysterogram (EHG) transformed into the frequency domain using fast Fourier transform (FFT).

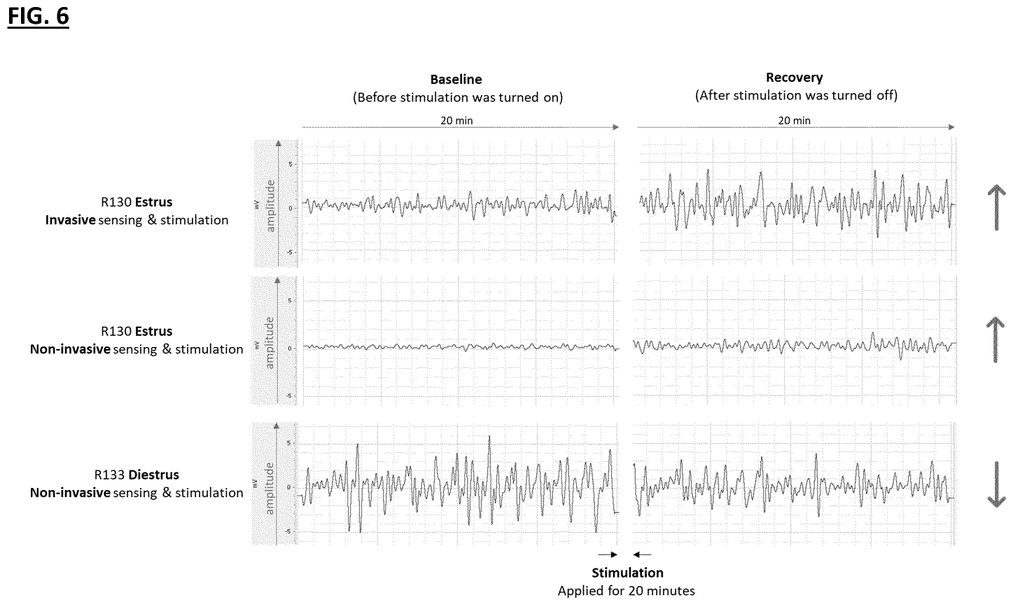

FIG. 6 demonstrates the effect of electrostimulation on uterine contractions in control rats (no endometriosis) during the Estrus, Proestrus and Diestrus stages of a rat hormonal cycle. Uterine contractions were recorded for a 20 minute period, electrostimulation was applied for 20 minutes, and then uterine contractions were recorded for a further 20 minutes. The graphs illustrate that in rats without endometriosis electrostimulation during the Estrus and Proestrus stages of the hormonal cycle caused an increase in the amplitude of contractions, whereas electrostimulation during the Diestrus stage of the hormonal cycle caused a decrease in the amplitude of contractions.

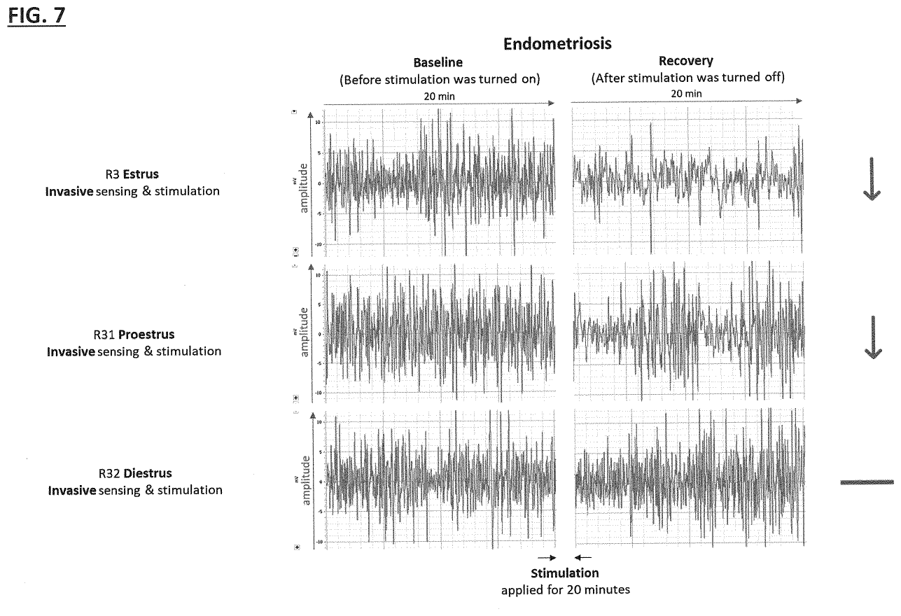

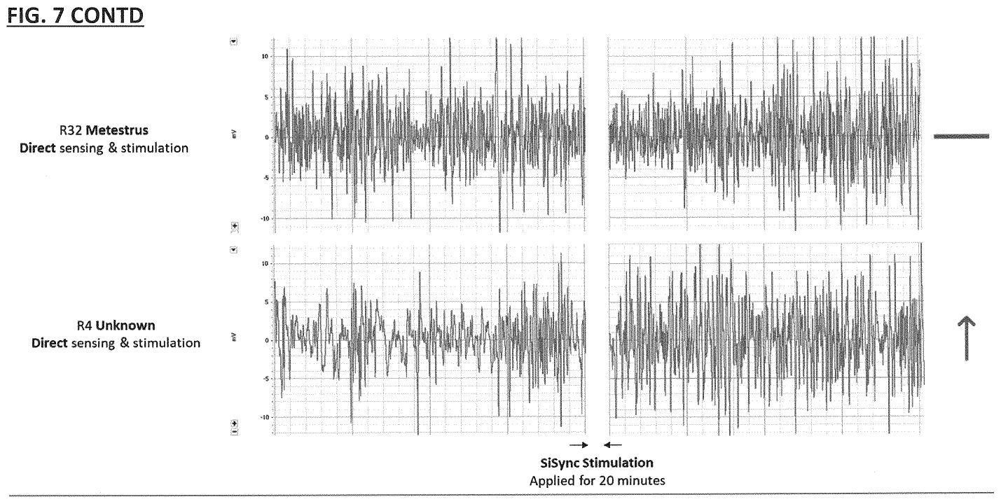

FIG. 7 demonstrates the effect of electrostimulation on uterine contractions in rats with endometriosis during the Estrus and Proestrus stages and Diestrus stage of a rat hormonal cycle. Uterine contractions were recorded for a 20 minute period, electrostimulation was applied for 20 minutes, and then uterine contractions were recorded for a further 20 minutes. The graphs illustrate that in rats with endometriosis electrostimulation during the Estrus and Proestrus stages of the hormonal cycle caused a decrease in the amplitude of contractions, whereas electrostimulation during the Diestrus stage of the hormonal cycle did not show this same effect.

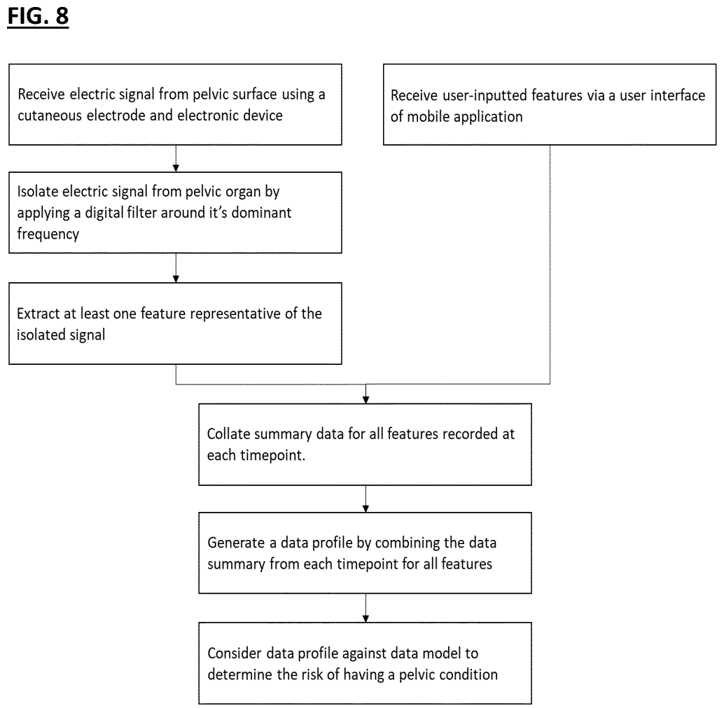

FIG. 8 is a flow chart illustrating a method of diagnosing a pelvic condition according to the invention.

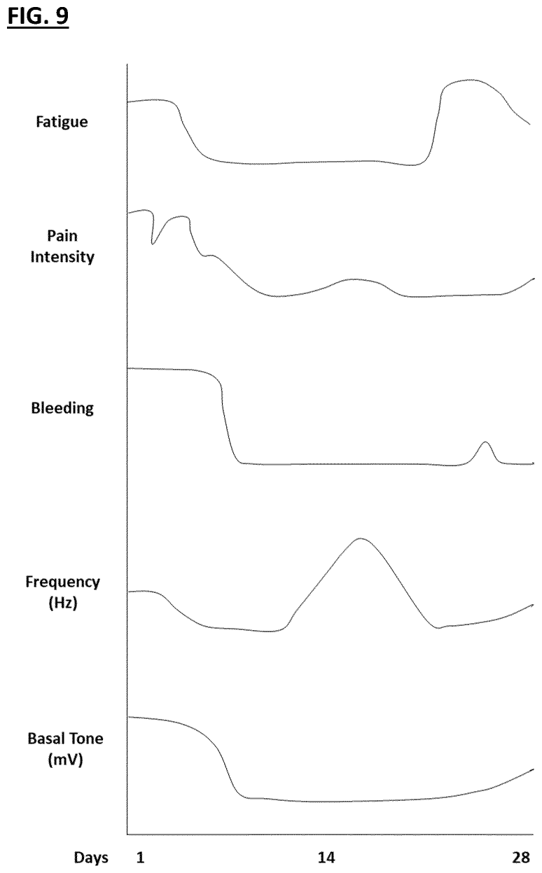

FIG. 9 illustrates an example of a data profile of a subject generated using two contractile parameters (contraction frequency, basal tone), and three non-electrical hormonal cycle parameter (fatigue, pain intensity, bleeding) mapped over a subjects 28-day hormonal cycle.

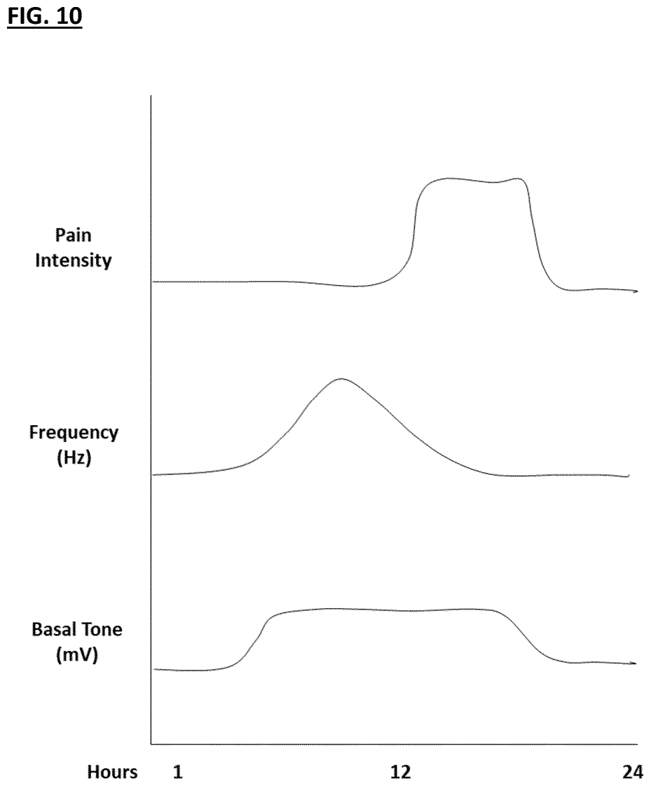

FIG. 10 illustrates another example of a data profile of a subject generated using two contractile parameters (contraction frequency and basal tone), and one non-electrical hormonal cycle parameter (pain intensity) mapped over a subjects 24-hour hormonal cycle.

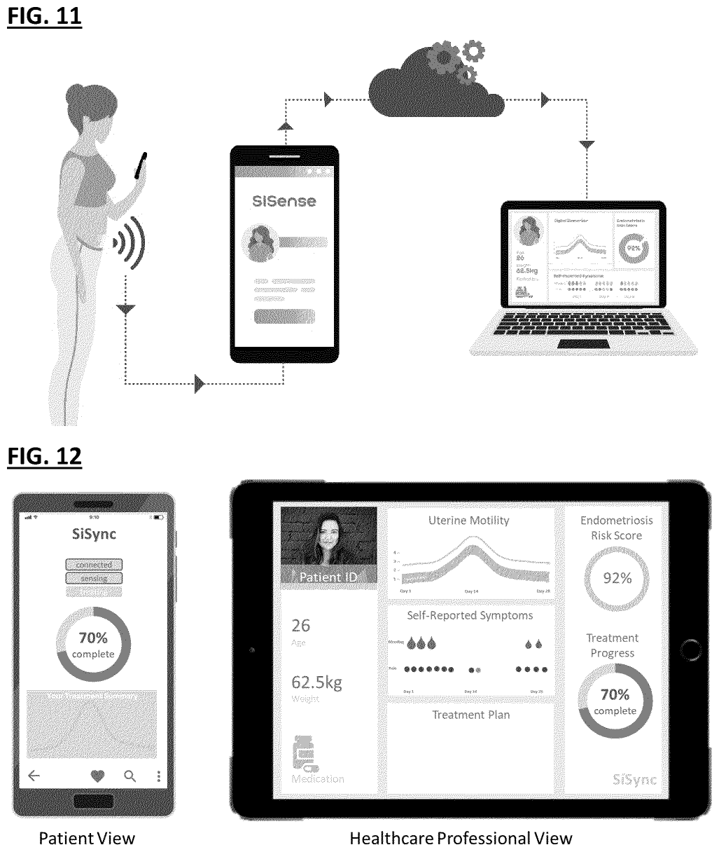

FIG. 11 illustrates a system for diagnosing a pelvic condition according to one embodiment of the invention which shows flow of data from the sensor placed on pelvic surface to the mobile application on the user's phone to the remote servers and to the personal device of the clinician.

FIG. 12 is an illustration of the summary data accessed from the remote server and presented to the patient and clinician on their respective personal computing devices.

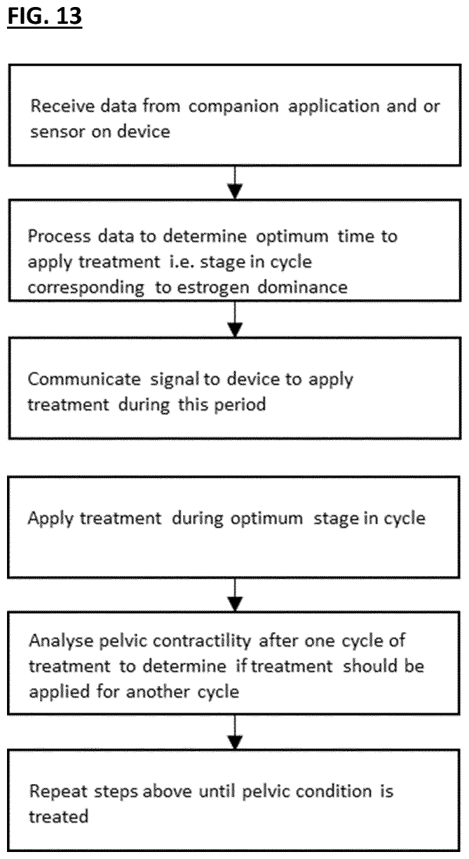

FIG. 13 is a flow chart illustrating a method of treating or preventing a pelvic condition according to the invention.

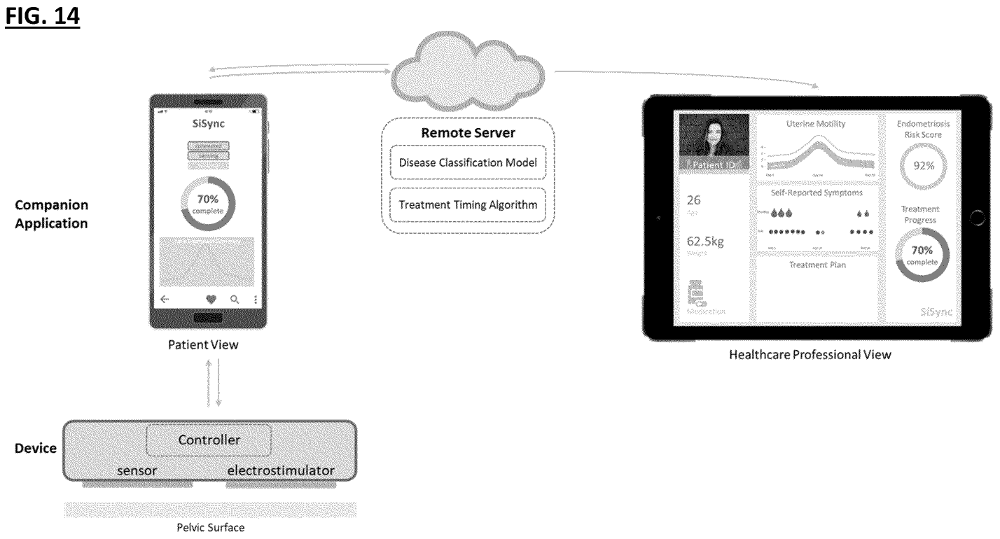

FIG. 14 illustrates a system for treating or preventing a pelvic condition according to one embodiment of the invention which shows flow of sensing data from the sensor placed on pelvic surface, to the mobile application on the user's phone, to the remote servers including the processor. The processor determines the status of a pelvic condition in the subject, calculates a specific stage of the hormonal cycle to apply stimulation, monitors the progress of the hormonal cycle in the subject, and actuates the electrostimulation device to apply electrostimulation during the calculated stage.

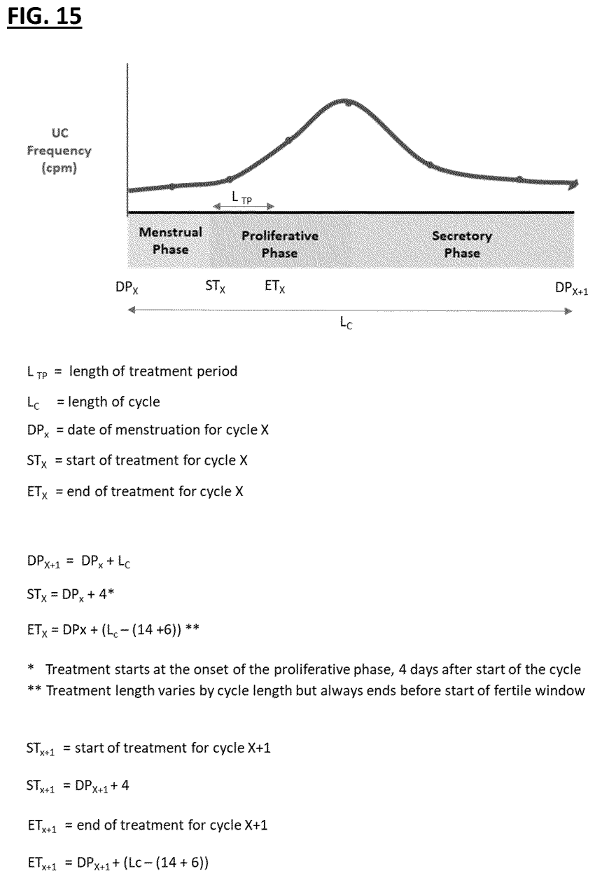

FIG. 15 illustrates a treatment protocol for a female subject determined to have endometriosis.

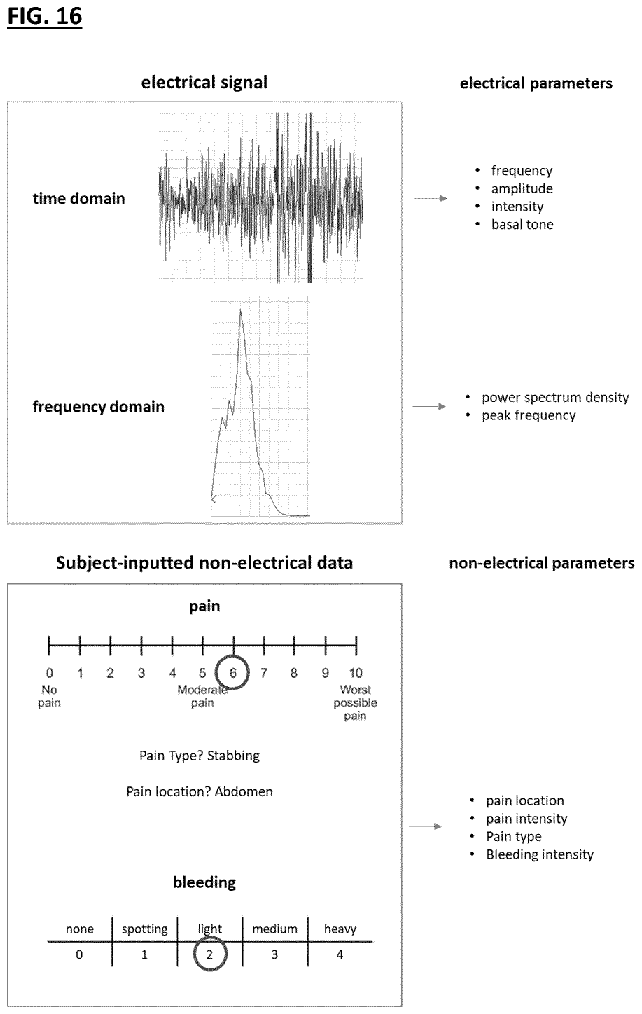

FIG. 16: Top—illustrates the extraction of a contractility parameter from electrical activity that employs a signal processing module to convert an electrical signal from the time domain into the frequency domain (power spectrum density v peak frequency). Bottom—non-electrical hormonal cycle parameter (pain) forming part of a data profile for a test subject

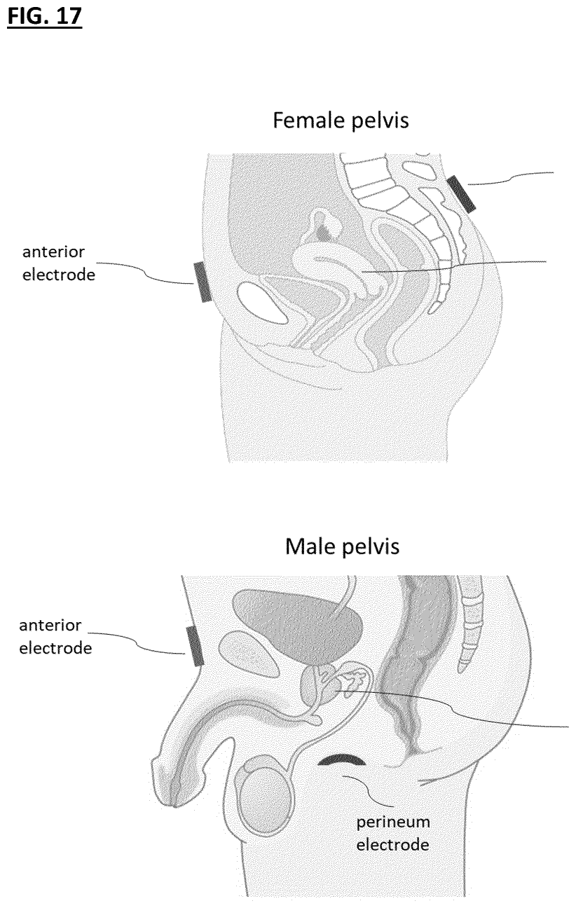

FIG. 17: Top—illustrates the placement of a non-invasive cutaneous sensor electrodes relative to target organ in a female subject. Bottom—illustrates the placement of a non-invasive cutaneous sensor electrodes relative to target organ in a male subject. The electrodes may be placed anteriorly or posteriorly.

FIG. 18 illustrates a closed loop sensing and stimulation system based on hormonal cycle.

FIG. 19 illustrates the comparison function of the system and process of the invention. Software embedded in the controller receives the electrical contractility parameters from the sensor and compares them to a healthy population template relative to that hormonal cycle stage (i.e. menstrual cycle day). The algorithm evaluates if the subject's reading is within a normal range for that timepoint. Based on this, the controller sends instructions to the electrostimulator to stimulate or not to stimulate a target pelvic structure that day.

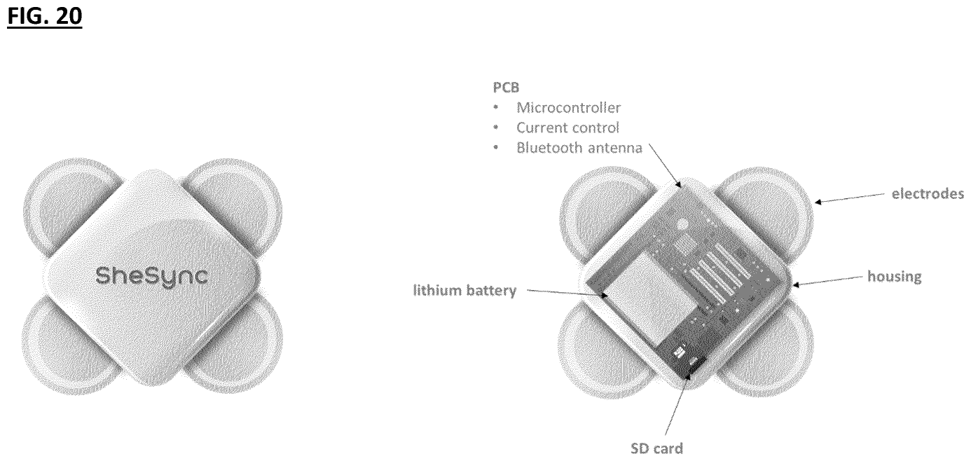

FIG. 20 illustrates a wearable sensing and stimulating module forming part of the system of the invention. The module is configured for cutaneous application in the pelvic region and comprises electrodes and a central housing that incorporates a battery, a PCB including a microcontroller, current control module and Bluetooth antenna, and a SD card.

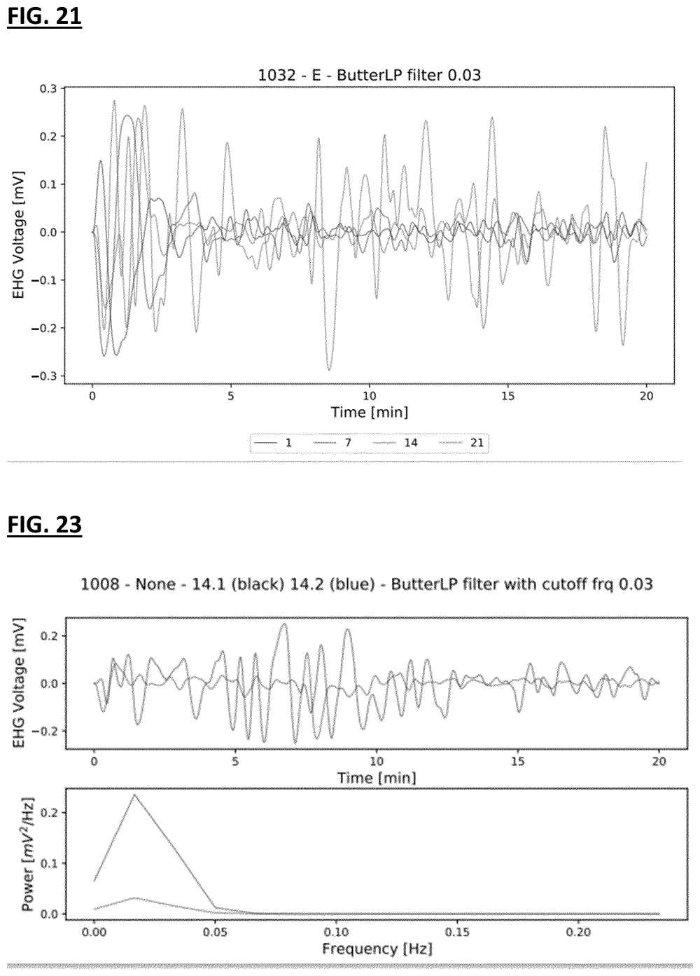

FIG. 21—All recorded signals (Day 1, 7, 14, 21) for a volunteer with endometriosis.

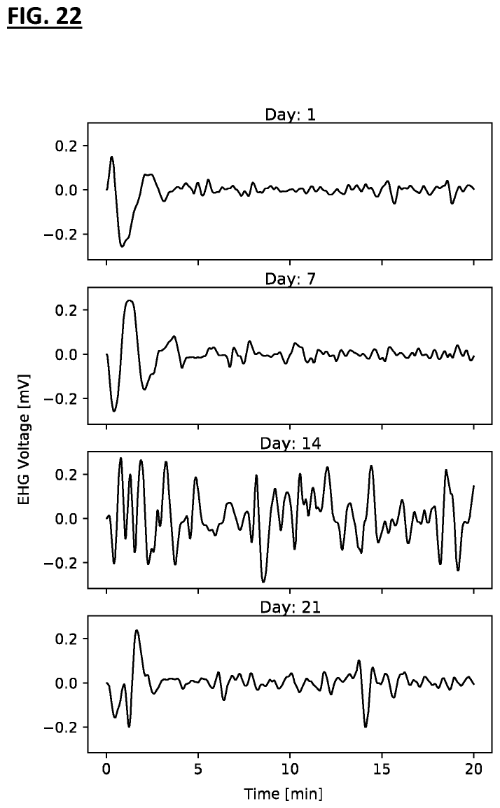

FIG. 22—All recorded signals (Day 1, 7, 14, 21) for a volunteer with endometriosis. This is the same volunteer as for FIG. 21.

FIG. 23—Recorded signals (top) and their power spectrum (bottom) for Day 14 and 15 of a healthy volunteer.

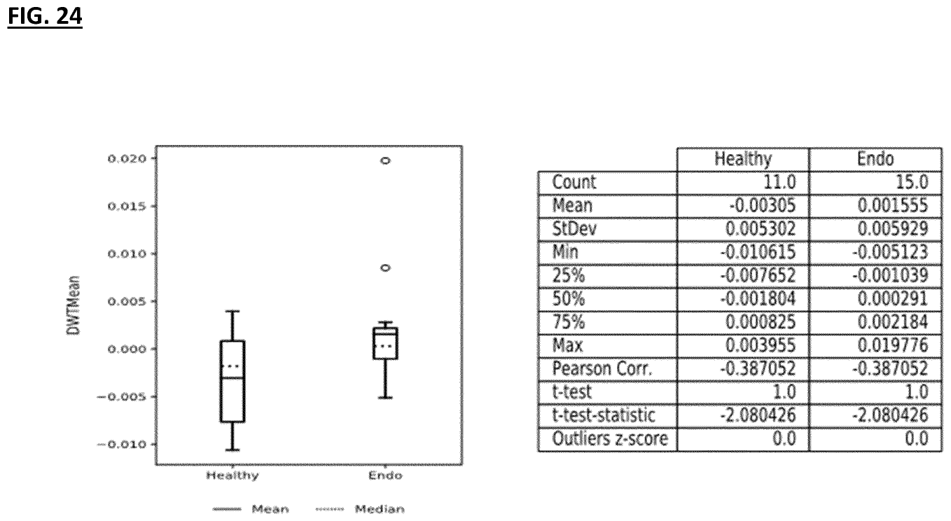

FIG. 24—Boxplot and statistical summary of DWTMean on day 14 for volunteers, healthy-no drugs (n=11) and endo-no drugs (n=15).

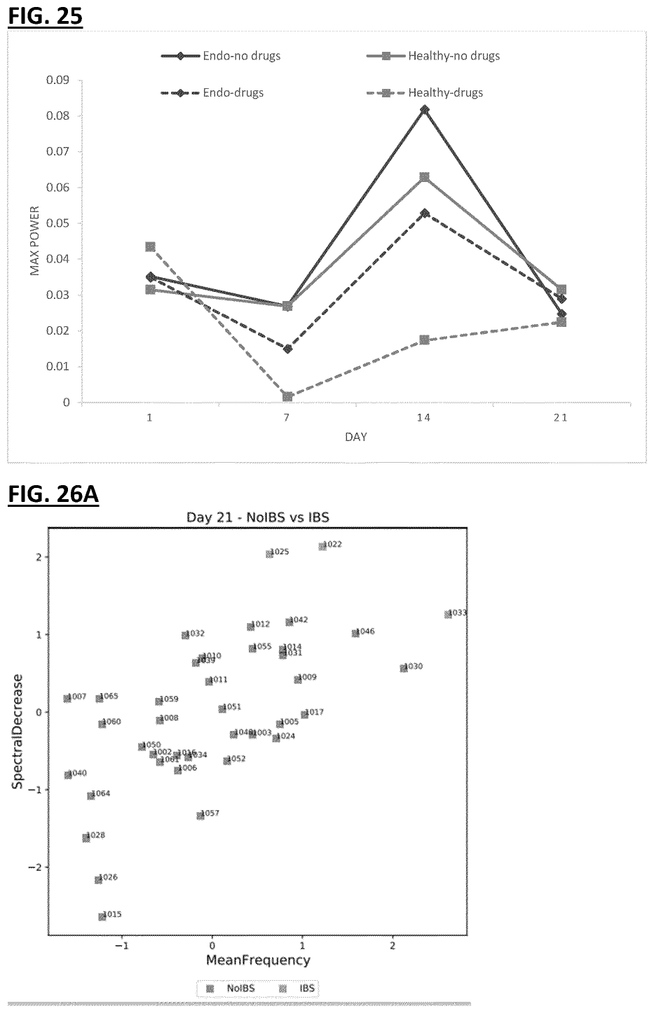

FIG. 25—Average “MaxPower” per day (1, 7, 14, 21) for volunteers and modulation of the signal with hormonal intervention, endo-no drugs (n=15), healthy-no drugs (n=11), endo-drugs (n=7), healthy-drugs (n=2).

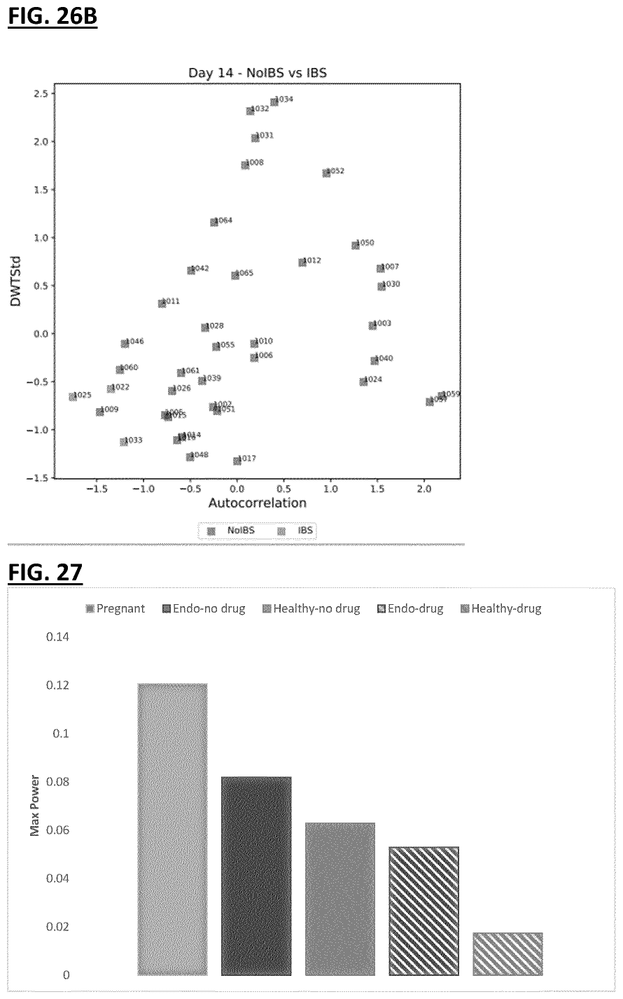

FIG. 26: Scatterplot of volunteers (n=39) with (red) and without (blue) IBS using the features (a) SpectralDecrease and MeanFrequency and (b) DWTStd and Autocorrelation

FIG. 27—Comparing MaxPower of volunteers at Day 14. Pregnant with miscarriage (n=1), endo-no drug (n=15), healthy-no drug (n=11), endo-drug (n=7), healthy-drug (n=2). When we look at the Pregnant+miscarriage Max Power at Day 14, it is elevated relative to all other volunteers—she has much more uterine motility which may impede implantation.

FIG. 28—Max Power of volunteers at various timepoints. Women with endometriosis surgically diagnosed due to pain (n=11), healthy volunteers (n=15), women with endometriosis surgically diagnosed due to fertility issues (n=4). Uterine motility at Day 14 is greatly reduced in those who have issues with fertility.

FIG. 29—Max Power of volunteers at various timepoints Fertility-no IVF (n=4), healthy-no drugs (n=11), fertility-IVF (n=1). Ovarian stimulation protocol in the volunteer undergoing IVF enhances ovulation relative to those volunteers with fertility issues who are not undergoing fertility treatment.

FIG. 30—Max Power of volunteers at various timepoints. Endo-no drugs (n=15), healthy-no drugs (n=11), hysterectomy (n=1). The ability to detect a signal due endometriotic lesions means that this technology will be able to monitor the effectiveness of treatments (surgery & medications) in terms of lesion removal/regression.

FIG. 31—Block diagram of one method of diagnosing endometriosis according to the invention.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, age, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure. Improvement may be observed in biological/molecular markers, clinical or observational improvements. In a preferred embodiment, the methods of the invention are applicable to humans, large racing animals (horses, camels, dogs), and domestic companion animals (cats and dogs).

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, camels, bison, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human. As used herein, the term “equine” refers to mammals of the family Equidae, which includes horses, donkeys, asses, kiang and zebra.

“Pelvic structure” is intended to include structures in the pelvic cavity that have a muscular component including the pelvic floor, bladder, rectum and descending colon, caecum, the uterus, fallopian tube, clitoris, vaginal, cervix, and ovaries in females and the prostate, penis, and testes in men. In one embodiment, the pelvic structure is a pelvic organ.

“Pelvic condition” refers to endocrine disorders and reproductive conditions that are associated with changes in contractility of one or more pelvic structures. “Reproductive conditions” may be pathological or non-pathological reproductive conditions or events including infertility, implantation failure (natural or during assisted reproduction), spontaneous miscarriage, or preterm birth. The methods and systems of the invention may be employed or configured to treat or prevent infertility and prevent or reduce the risk of unwanted reproductive events such as implantation failure, spontaneous miscarriage, or preterm birth in women.

“Endocrine disorder” or “endocrine condition” refers to diseases relating to the endocrine glands of the body which typically results in a hormone imbalance. Examples originating from glands in the pelvic cavity include endometriosis, adenomyosis, endometritis, chronic pelvic pain, benign prostate hyperplasia, prostatitis, interstitial cystitis, pelvic inflammatory disease, irritable bowel syndrome, inflammatory bowel disease, heavy menstrual bleeding, dysfunctional uterine bleeding, hormone-dependent cancers of the pelvic (ovarian, uterine, endometrial, prostate, testicular, bladder), polycystic ovary syndrome, follicular maturation arrest, anovulation, dysmenorrhea, anovulation, infertility, uterine leiomyoma, precocious puberty, endometritis, erectile dysfunction, incontinence (fecal incontinence, stress urinary incontinence, urge incontinence, mixed incontinence), pelvic floor myalgia, pelvic floor dysfunction, dysuria (painful urination), dyspareunia (pain during intercourse), dyschezia (painful defaecation), dysorgasmia (painful ejaculation)

“Contractility parameter” as applied to a pelvic structure is intended to mean the motility, tone, occurrence, frequency, amplitude, strength, direction, power, power density, pattern, duration, periodicity, dominant frequency, peak to peak, or area under the curve of contractions in the pelvic structure. Preferably, the contractility parameter is selected from frequency, amplitude, and basal tone.

“Slow wave electrical contractility”. In any aspect, the contractility parameter may be a slow wave electrical contractility parameter such as slow wave electrical contractility frequency. Slow wave contractility is generally caused by an inner smooth muscle layer of a target organ, for example the inner endometrial SM layer in the uterus or the myogenic SM layer in the prostate. Slow wave contractility in the uterus and caecum is generally measured in the 0.00 to 0.05 Hz range.

“Status” as applied to a pelvic condition in a subject should be understood to mean positive or negative diagnosis of the pelvic condition, risk of development or occurrence of the pelvic condition, response to the pelvic condition to treatment, severity of the pelvic condition, or any other clinically useful information relating to the pelvic condition. Specific examples include diagnosis of endometriosis, IBD, risk of miscarriage or infertility in a female (generally a non-pregnant female), and diagnosis of a prostate endocrine disorder (e.g. prostate cancer or BPH) in a male.

“Sensing module” means a sensor that can detect a contractility parameter of a target pelvic structure. The sensing module is generally an external sensor. The sensing module may take the form of a patch configured for cutaneous attachment to the subject. The sensing module may be wearable. The sensing module may be configured for sub-cutaneous application. The sensing module may be an electrical sensor configured to detect electrical activity of the pelvic region. The sensing module maybe configured to transmit sensing data wirelessly, for example to a mobile device or computer. The sensing module may include one or more sensing electrodes that may be spaced apart. The sensing module may be placed on an abdomen of a subject is proximity to a target structure. Examples of suitable electrical sensing modules include the Biosignalsplux Solo kit and the Biosignalsplux Electrogastrogaphy (EGG) sensor, both made by Wireless Signals SA.

“Plurality of time points during the subjects hormonal cycle” means at least two time points, and typically at least 5, 10, 15, 20 or 25 time points. The time points are generally spaced apart during the hormonal cycle. Usually, at least one time point occurs in each stage of the hormonal cycle, for example at least 2, 3, 4, 5, or 6 time points per stage of the hormonal cycle. The measurements taken at the plurality of time points map the variable being measured over the course of the cycle. The variable may be a contractile parameter (frequency or intensity), or a non-contractile hormonal cycle parameter (bleeding, pain, or fatigue). The data collected at each timepoint may be processed into a representative data summary. The timepoints may be equally spaced over the extended recording period, for example daily. After recordings are completed, the signal across the hormonal cycle may be represented by mapping the summary data generated (electric and user-inputted) at each timepoint, to create a data profile for that subject. In a non-pregnant female, the time points may be at 1, 7, 14 and 21 days of their menstrual cycle (+1-1 or 2 days). For females with irregular hormonal cycles, a measurement may be taken at day 13, 14 and 15, compared, and one of the measurements employed (for example the measurement with the highest Max Power). Measurements of electrical activity (e.g. signals) are generally recorded for at least 10, 15, 20 or 25 minutes.

“Subjects hormonal cycle” as applied to a female subject refers to the cyclical changes in a woman's body during reproductive years caused by the complex interaction of hormones: luteinizing hormone, follicle-stimulating hormone, and the female sex hormones estrogen and progesterone. The stages of a female hormonal cycle are the follicular phase, the ovulatory phase and the luteal phase. In animals with estrus cycles, the proestrus stage is equivalent to the follicular phase, the estrus stage is the equivalent of the ovulatory stage, and the diestrus stages are equivalent to the luteal phase. As applied to a male mammal, the term refers to cyclical hormonal changes over a period of time (e.g. 24 hours) and changes that occur as males age (i.e. andropause). In one embodiment, the invention comprises stimulating a target pelvic structure during a specific stage of the subject's hormonal cycle with a view to normalising pelvic structure contractions. In females of reproductive age with an endocrine disorder such as endometriosis, stimulation is typically carried out during the follicular stage.

“Signal processing module” refers to an apparatus configured to receive electrical activity signals from the sensing module and process the signal. The signal may be processed to amplify and/or digitize the signal. Digitization of the signal may be performed by an analogue to digital converter. The signal may be processed extract a signal (e.g. an electrical contractility parameter) that is representative of a target pelvic structure. In some embodiments, this is achieved by applying a digital filter corresponding to the dominant or characteristic frequency of that structure. Alternatively, the digitized signal can be transformed into the frequency domain, and the contractility structures are isolated from the overall pelvic EMG signal for example by dividing the frequency spectrum into segments corresponding to the characteristic frequency of each pelvic structure. In some embodiments, signals representative of the uterus, colon, bladder, prostate and pelvic floor are isolated within the frequencies 0-0.05 Hz, 0.2-0.4 Hz, 0.1-5 Hz, 0.06-0.11 Hz and 20-500 Hz respectively. In some embodiments, the signal is processed to isolate slow wave electrical contractility characteristic of the target organ. In many pelvic organs of interest, the slow wave activity has a frequency in the 0.00 to 0.05 Hz range, typically 0.01 to 0.03 or 0.01 to 0.02 Hz. The slow wave signal is characteristic of inner smooth muscle of the target organ, for example the endometrial SM layer in the uterus and the myogenic SM layer in the prostate. In some cases, the methods and systems of the invention can include algorithmic processing of the isolated signal to compensate for body position and artefact coming from other parts of the body (heart, GI tract, respiration, skeletal muscle) and to further extract parameters of interest (e.g. frequency, basal tone, amplitude). These methods can include linear modelling, digital filtering, spectral analysis and statistical analysis. The quality of the signal can be further enhanced by recording the signal over a prolonged period at each timepoint, for example 30 minutes, and averaging the signal to reduce the signal to noise ratio.

“Data profile” refers to a plurality of measurements of one or more contractility parameters mapped over a defined period of time, for example over the duration of a hormonal cycle (e.g. menstrual cycle in a non-pregnant female). The data profile may include one or more non-electrical hormonal cycle parameters mapped during the same time period, examples include hormonal cycle parameters such as bleeding, fatigue, pain intensity and pain occurrence. Generally in a data profile comprising more than one variable, the different variables will be mapped at the same time points. Examples of data profiles are provided in FIGS. 9 and 10. Generally the data profile comprises at least one contractility parameter (for example, 1, 2 or 3) and, optionally, at least one non-electrical hormonal cycle parameter (for example at least 1, 2, 3, 4 or 5). In one embodiment, the contractility parameter is converted from the time domain into a frequency domain.

“Reference data profiles” refers to a data profile of a subject with a known pelvic condition status, for example when the system or method is for detecting endometriosis in a subject, the reference data profile may be a data profile from a subject positive or negative for the condition. Generally the subjects data profile is correlated with pelvic condition status by employing a classification model generated using reference data profiles from a population of subjects with known pelvic condition status, for example positive disease, negative disease, risk of developing disease, and severity of disease. Generally, when the subject's data profile comprises more than one variable mapped over time, the reference data profiles against which the subject's data profile is compared will all include the same variables mapped over time. Comparison of the subject data profile with the reference data profile or profiles generally employs a computational model, which may be multiple linear computational model. Various methods may be employed to match a subject's data profile with one of the reference data profiles including mathematical modelling or pattern recognition. In one embodiment, the comparison step may be performed by mathematical modelling using ‘Linear discriminant analysis’ and ‘nearest neighbour Euclidian distance minimisation’, using a subset of the chemical growth responses. Other methods of matching or correlating a query data profile with one or more reference data profiles involves simple Euclidian matching or hierarchical cluster analysis. In one embodiment the reference data profile is from the same subject obtained previously, for example before treatment. This allows a subject or physician to monitor a pelvic condition over time to determine changes in the pelvic condition in the subject (for example before or after treatment). The reference data profile in the context of determining fertility and in the context of IVF-related applications is generally obtained from one or more healthy fertile women. The systems and methods of the invention may also be employed to determine pelvic condition status of a subject relative to a cohort of people, for example relative to a population defined by age, geography, habits (e.g. alcohol use, smoking) ethnicity, race, sex, number of pregnancies, or any combination thereof (for example woman in the 20-30 age bracket) any other cohort.

“Non-electrical hormonal cycle parameter” refers to a hormonal cycle related parameter in the subject that is not electrical. Examples include pain intensity, pain location, pain type, bleeding, urination patterns, bowel patterns, mood, bloating, fatigue, weakness or impact to daily life. Pain can include pelvic pain, back pain, upper abdominal pain, vaginal pain, labia pain, perineum pain, breast pain, pain during intercourse, pain after intercourse, pain during ejaculation, pain during urination or pain during defecation, chills, fever or lack of energy. Bleeding patterns include menstrual bleeding, spotting, blood in semen or blood in urine. Urination patterns include increased or decreased frequency or flow or feeling of needing to urinate. Bowel patterns includes constipation, diarrhoea, an increased frequency, or a decreased frequency. Impact to daily life includes missed days at work, school, inability to exercise or complete household chores. Measurements of these parameters may be input by the subject, for example using a user interface of a mobile phone or a computer.

“Non-electrical, non-hormonal cycle parameter”: The data profile may also include a non-electrical, non-hormonal cycle parameter. These parameters are subject phenotype parameters, for example age, sex, reproductive status, hormonal cycle status, previous diagnoses or conditions, family history, medical records, medical imaging, BMI, symptoms and medication. The use of one or more of these variables in a data profile can be used to inform the reference data profiles employed in determining pelvic condition status in the subject. For example, if the subject is female and age 35, a specific classification model may be employed to determine and provide an output of pelvic condition status.

“Pelvic structure stimulating module” is an apparatus configured to stimulate a target pelvic structure to module at least one contractility parameter of the pelvic structure. In the embodiments described herein, an electrostimulation device is employed. The device may be configured to emit electrical pulses of 0.1 to 20 mA. The device may be configured to emit electrical pulses with a pulse width of 500 μs to 20 ms. The device may be configured to emit electrical pulses at a frequency of 0.1 to 50 Hz. Stimulation may be applied for 30-60 minutes at a time. The device may comprise one or more or an array of electrodes. The module may be configured for cutaneous application, and stimulation of the pelvic structure from the surface of the subjects body. The stimulating module may be configured to wirelessly receive signals from a remote location, for example a mobile communications device or a computer. The signals may be instructions relating to the type and extent of the electrical stimulation, and the timing of the electrostimulation. Stimulation of the target pelvic structure may also be achieved using magnetic waves, high-intensity light waves, shockwaves waves, high-energy laser radiation or electroacupuncture. Typically, the stimulation module is configured to apply a stimulation configured normalise contractility of the pelvic structure (e.g. modulate the contractility parameter so that it resembles a corresponding contractility parameter from a person negative for the disease. Generally, this involves a stimulation configured to normalise contractions or reduce the frequency, amplitude, intensity or basal tone of the contractions.

“Monitor the subject's hormonal cycle”: The system and methods of the invention involve in one embodiment monitoring of the subjects hormonal cycle. This allows treatment of the subject at one or more specific stages of the hormonal cycle. Monitoring comprises taking measurements during the hormonal cycle of at least one contractility parameter or another variable relevant to the hormonal cycle, for example temperature, date of last menstruation, or cervical discharge status. The contractility parameters are sensed by the sensing module, and the other variables may be input by the user, and the processor may be configured to monitor progression of the hormonal cycle from the measurements received, and then actuate the simulation module at a specific stage during the hormonal cycle.

“System” in the context of determining status of a pelvic condition comprises a sensing module, optionally a signal processing module, and a processor. The system may also include software for a computational device, especially downloadable software suitable for use with a mobile communications device such as a mobile phone. The sensing module or signal processing module may be configured to transmit data to the computation device wirelessly. The software may be configured to cause the communication device receive data from the sensing or signal processing module, optionally store the data, transmit the data to a processor (for example a processor in a remote location), and receive data from the processor relating to the status of a pelvic condition in a subject, and display some or all of the data. The processor may be configured to transmit data relating to the status of the pelvic condition to another location, for example a computational device in a hospital or physician's office.

“System” in the context of treating or preventing a pelvic condition additionally includes a pelvic structure stimulating module, for example an electrostimulation device. The module may be configured to receive treatment instructions from a remote location, for example a mobile communications device. The processor may be configured to generate treatment instructions, including treatment parameters including the duration, intensity, and stage of hormonal cycle when the treatment is to be applied. The software may be configured to cause the mobile phone receive the treatment parameters from the processor and transmit the treatment parameters to the stimulation module.

“Wearable device” refers to a device comprising a sensing module, optionally a signal processing module, and a pelvic structure stimulation module. The device is wearable and may be provided in the form of a patch that can be applied to the subject cutaneously. The device generally includes a wireless communication module configured to transmit data to a remote location, and receive data from a remote location. The device may include one or more sensing or treatment electrodes. The device may include a power source (for example a battery) operatively connected to any of the modules of the device. The device may include a controller (e.g. a microcontroller) operatively connected to the pelvic structure stimulation module and optionally the power source.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

Materials and Methods

Animal Model

Female Sprague Dawley rats weighing 200 to 250 g were housed at 23° C. in 12-hour light/dark cycle with food and water ab libitum. They were randomly assigned to Endometriosis or Sham group with 8 animals per group. The Animal Care Research Ethics Committee (ACREC) at National University of Ireland, Galway approved all procedures. Animals were handled (5 min/d) for 7 days prior to beginning the experiments to reduce manipulation stress, and vaginal cytological smears carried out to verify reproductive cycles.

Induction of Endometriosis

Endometriosis was induced surgically under isoflurane anaesthesia, based on the method by Vernon and Wilson (1985). The distal 2 cm of the right uterine horn was removed and immersed in warm (37 deg) sterile saline. The endometrium was exposed by opening the uterine horn lengthwise with a sterile scissors. Four pieces of uterine horn 5 mm2 were cut using a biopsy punch. The implants were sutured with the serosal surface next to the mesenteric vessels of the small intestine and the endometrial surface exposed to the peritoneum. In sham-operated groups, the right uterine horn was explanted, and 4 sutures were attached to the mesentery of the intestine without uterine implants. The peritoneal cavity was kept moist with copious amounts of saline solution throughout the surgery to reduce adhesions. The endometriosis was allowed to progress for 56 days following the induction surgery before electrohysterogram (EHG) recordings and electrostimulation tests were completed.

Electrohysterogram (EHG) Recordings

A laparotomy was performed under isoflurane anaesthesia. For direct measurements, a bipolar needle electrode (AD Instruments) were inserted into the myometrium (the distance between the two electrodes was 8 mm). For non-invasive measurements, an abdominal skin incision was created and a bipolar disk electrode pair (MDE GmbH Walldorf, Germany) was placed subcutaneously above the uterus (the distance between the two electrodes was 20 mm). The basal contractility of the uterus was detected for 60 minutes. The electric signals were recorded an analysed by an online computer and amplifier system (AD Instruments PowerLab and Quad BioAmplifier). All analogue signals were converted to a digital signal at a sample rate of 1000 Hz.

During the recording animals were maintained under isoflurane anaesthesia. When the experiments were completed, animals were sacrificed per Directive 2010/63/EU. A digital filter was applied to the recorded signals (low pass 0.1 Hz). To compare EHG between groups (endometriosis and sham) exploratory statistical analysis was computed on raw signals (see Table 1). They were further analysed by fast Fourier transformation (FFT) where the frequency of the electrical activity was characterised in Hz, and the magnitude of the activity was described as power spectrum density (see FIGS. 1-4).

Electrostimulation Tests

A second bipolar electrode made of Teflon-insulated multistranded stainless steel was inserted into the myometrium, spaced 10 mm from the sensing electrode. For non-invasive electrostimulation, a bipolar disk electrode pair (MDE GmbH Walldorf, Germany) was placed subcutaneously above the uterus (the distance between the two electrodes was 20 mm). Baseline EHG was recorded for 20 minutes (as previously described). The electrode was connected to a pulse generator (Multichannel Systems: Stimulus Generator 4002) which was pre-programmed with constant current square wave pulses at 1-2 mA, 2 msec/pulse, 2-15 Hz. Electrostimulation was applied for 20 minutes before disconnecting the electrodes from the pulse generator and recording the recovery EHG for a further 20 minutes.

During the recording animals were maintained under isoflurane anaesthesia. When the experiments were completed, animals were sacrificed per Directive 2010/63/EU A digital filter was applied to the recorded signals (low pass 0.1 Hz). Results were analysed by fast Fourier transformation (FFT) where the frequency of the electrical activity was characterised in Hz, and the magnitude of the activity was described as power spectrum density (FIG. 5). The raw signal was compared in FIGS. 6-7 to demonstrate the effect of electrostimulation at different points in the hormonal cycle.

Results

FIG. 1 illustrates that uterine contractility parameters measured at all stages of the hormonal cycle using an electrical sensor can be used to distinguish between rats with and without endometriosis. Uterine contractility is represented by power spectrum density at peak frequency.

FIGS. 2 and 3 illustrate that the differences in uterine contractility between the rats with and without endometriosis are especially pronounced during the proestrus and diestrus stages of the hormonal cycle.

Endometriosis and sham animals can also be distinguished using other contractility parameters as indicated in Table 1 below:

TABLE 1

| | Endometriosis | | Sham Group | | | | |
|---|---|---|---|---|---|---|---|
| Features | Mean | Variance | Mean | Variance | Difference | p Value | Significance |
| Peaks (mV) | 0.0026 | 2.5e−7 | 0.0016 | 6.6e−7 | 0.0009 | 2.58e−5 | Yes |
| amplitude | | | | | | | |
| Troughs (mV) | −0.0020 | 2.7e−7 | −0.0011 | 5.4e−7 | 0.0008 | 4.92e−5 | Yes |
| basal tone | | | | | | | |
| Peaks Rate (per minute) | 1.61 | 0.061 | 1.78 | 0.048 | 0.1740 | 0.0182 | Yes |
| frequency | | | | | | | |
| Area under Peaks (mVs) | 22.67 | 41.24 | 13.28 | 30.09 | 9.3920 | 5.26e−6 | Yes |
| intensity | | | | | | | |

Table 2 illustrates a data profile for a subject comprising electrical contractility parameters determined at four time points T1 to T4 and non-electrical hormonal cycle parameters (pain location, pain intensity, pain type and bleeding intensity) determined at the same time points.

TABLE 2

| timepoint | | T1 | T2 | T3 | T4 |
|---|---|---|---|---|---|
| Electrical | Frequency | 0.02 | 0.05 | 0.067 | 0.002 |
| parameters | (Hz) | | | | |
| | Amplitude | 1.4 | 2.0 | 1.6 | 1.4 |
| | (mV) | | | | |
| | Intensity | 2.8 | 6.7 | 3.4 | 2.9 |
| | (mVs) | | | | |
| | basal tone | 2.8 | 3.7 | 2.7 | 2.8 |
| | (mV) | | | | |
| Non-electrical | Pain location | abdomen | abdomen | — | — |
| parameters | Pain intensity | 6 | 4 | 0 | 0 |
| | Pain type | stabbing | stabbing | — | — |
| | Bleeding | 4 | 2 | 0 | 0 |
| | intensity | | | | |

FIGS. 5 to 7 demonstrate that contractility of the uterus in mammals can be modulated using electrostimulation, and that the effect of electrostimulation is informed by the hormonal status of the animal. For example in FIG. 6, application of electrostimulation to control rats (no endometriosis) during proestrus (equivalent to the follicular stage of the hormonal cycle in humans) increased contractile activity, whereas application of electrostimulation to rats with endometriosis during proestrus decreased or normalised contractile activity, as indicated in FIG. 7. This is summarised in Table 3 below:

TABLE 3
Summary of effects of SiSync Electrostimulation over
the hormonal cycle:

| | | | |
|---|---|---|---|
| Human Cycle | Follicular | Ovulation | Luteal |
| Rat Cycle | Proestrus | Estrus | Diestrus (inc. Metestrus) |
| Control | [symbol] | [symbol] | [symbol] |
| Endometriosis | [symbol] | [symbol] | [symbol] |

Clinical Data

Data Sources

The following data was collected from volunteers who consented to the study:
1. Uterine signal—This is an electro-hysterography (EHG) signal recorded via “Biosignalplux solo” device which is CE marked for research purposes. This is numerical and time ordered data.
2. Self-reported symptoms—This data is collected through a daily questionnaire completed by each volunteer. The questions touch on a variety of topics like pain, bleeding patterns, overall health, medication, etc. This is mostly ordinal and categorical data.
3. Other patient data—This data is collected through a pre-study questionnaire and includes information like height, weight, age, nationality, etc. This is mostly numerical and categorical data.

Study Recruitment

In the initial study, data was collected from 39 volunteers. All the volunteers are divided into four groups that are defined below. Each group is further divided depending on whether volunteers are on a hormonal intervention.
1. Healthy: Self-selected volunteers, have a normal menstrual cycle and do not have pain throughout the cycle.
2. Endo: Volunteers who are surgically diagnosed with endometriosis and have pain throughout the cycle.
3. Others: Volunteers who are medically diagnosed with endometriosis or who think they have endometriosis and have pain throughout the cycle.
4. Hysterectomy: Volunteers who do not have a uterus.

As detailed in Table 4, 13 women are healthy and 22 have endometriosis. Three women have been categorised as “others” for a variety of reasons listed in Table 5.

TABLE 4

| | No | | |
|---|---|---|---|
| | Drugs | Drugs | Total |
| Healthy | 11 | 2 | 13 |
| Endo | 15 | 7 | 22 |
| Hysterectomy | 1 | | 1 |
| Others | 2 | 1 | 3 |
| Total | 29 | 10 | 39 |

All vounteers by group and hormal intervention (n = 39)

*Drugs indicate hormonal interventions including Mirena, Progesterone Pill, Combined Contraceptive Pill and GnRH agonist. Some were on more than one hormonal intervention.

TABLE 5

| ID | Reason for categorisation as “others” |
|---|---|
| 1009 | Not surgically diagnosed but only medically diagnosed. |
| 1025 | Not surgically or medically diagnosed but given her |
| | symptoms she thinks she has endometriosis. |
| 1064 | Not surgically or medically diagnosed but given her |
| | symptoms she thinks she has endometriosis. |

Volunteers (n = 3) that have been categorised as “others” and related reasons.

Volunteers with endometriosis were recruited with the support of the Endometriosis Association of Ireland and EndoAware and as such are mostly Irish and British. The healthy volunteers are of various nationalities and reflective of the diversity of the research team who requested that their families and friends volunteer for the study. The groups are well matched in terms of age (29-33 years) and well represented in terms of distribution of weight.

TABLE 6
Nationality

| | | No Drugs | Drugs |
|---|---|---|---|
| Healthy | Irish | 5 | 1 |
| | Indian | 2 | |
| | Pakistani | 2 | |
| | French | 1 | |
| | Italian | 1 | |
| | Spanish | | 1 |
| Endo | Irish | 13 | 6 |
| | British | 2 | 1 |

Age

| | No Drugs | Drugs |
|---|---|---|
| Healthy | 31 | 33 |
| Endo | 33 | 29 |

Overweight

| | |
|---|---|
| Yes, BMI ≥ 25 | No, BMI < 25 |

| | No Drugs | Drugs | No Drugs | Drugs |
|---|---|---|---|---|
| Healthy | 4 | | 7 | |
| Endo | 5 | 2 | 10 | 5 |

Demographics of healthy (n = 13), endo (n− = 22): (a) Nationality (b) Age (c) Weight

In an extension study a further 5 volunteers with endometriosis diagnosed due to fertility issues (rather than pelvic chronic pain) were recruited, one of whom was undergoing ovarian stimulation for IVF treatment.

Data Collection, Pre-Processing, and Filtering

Data Collection: Uterine signals are collected via a CE marked portable device for research purposes “Biosignalplux solo”. Volunteers are asked to record the signal during four key days of their menstrual cycle: day 1, day 7, day 14, and day 21. Signals are recorded for 30 minutes. The volunteers are asked to lie still during the recording sessions and to collect signals, if possible at the same time of day for each recording session. An example of the four recorded signals for a given volunteer is shown in FIG. 22.

Pre-processing: Signals are pre-processed through several steps before analysis. First, signals are transformed using a “transfer function” that scales the signal to fit in the range of ±0.25 millivolts. Then, the first and last 30 seconds of signals are removed. Finally, signals are cut off at the 20th minute. Signals that are shorter than 20 minutes are discarded.

Filtering: The Biosignalplux solo device (and EMG sensors) collects signals between 0.01591-0.1591 Hz (˜0.96 cpm-9.5 cpm; cpm=contraction per minute). In all the analysis carried out in this report raw signals are filtered using a Butterworth low-pass filter with cut-off frequency equal to 0.03 Hz. The rationale is that we are interested in the non-pregnant uterus' contractile activity that is best described by slow waves. This approach was validated in our pre-clinical studies.

Data labelling: With respect to the key days of the menstrual cycle, the first day of menstrual bleeding is considered Day 1 of the cycle—estrogen levels are low and bleeding is typically heavy. By day 7, bleeding usually stops, estrogen levels are rising and the dominant follicle containing an egg is growing. Day 14 is the day when an egg is released from the ovary, and it is referred to as the day of ovulation. Day 21, the egg is joined by a sperm when travelling in the fallopian tube and after fertilisation the resulting embryo implants in the uterine wall. If however, you are not pregnant, estrogen levels decline again and the uterine lining will prepare to shed.

However, menstrual cycles are highly individual and may be longer or shorter than the classical 28-day cycle. Therefore, ovulation may happen early or late with respect to the 14th 15 day of the cycle. For this reason, women who have stated that they have an irregular cycle have been asked to record their signal during the 13th, 14th, and 15th days of their cycle. These signals were compared and the one having the highest MaxPower retained. An example of two recordings (Day 14 and 15) for a healthy volunteer is shown in FIG. 23.

Signal Feature Analysis

DWT Mean—(average value of the coefficients of a discrete wavelet transform computed using Haar wavelet) showed a statistically significant difference between the healthy volunteers and women with endometriosis on Day 14.

Plotting the average Max Power values for healthy women versus women with endometriosis over the 4 time points, a pattern like that of uterine motility across the hormonal/menstrual cycle emerges. The signal for women with endometriosis is elevated around the period of ovulation relative to healthy volunteers. Hormonal intervention reduces the signal for both healthy volunteers and women with endometriosis. This demonstrates the utility of this signal as a non-invasive digital marker for uterine motility.

It will be appreciated that many different filtering and mathematical techniques can be used to isolate and identify the one or more electrical contractility parameter measurements to generate the data profile of the subject comprising the isolated electrical contractility parameter measurements representative of the target pelvic structure. The system and method of the present invention makes use of the fact that the electrical contractility parameter is a signal comprising a slow wave contractility frequency electrical signal measurement originating from a smooth muscle or organ characterised by a low frequency content. Low frequency content of the uterus and caecum can be characterised by the frequency range of 0.00 to 0.05 Hz. The system isolates and identifies these low frequency signals to build a profile of the subject which can be compared with other profiles to provide a diagnosis of the health of an organ in the pelvic area. In addition the generated profile can detect conditions in a subject that heretofore was asymptomatic of that condition in a simple and non-invasive manner. The system can be further configured to provide an estimate or calculate a prediction value of whether a health condition is likely to develop based on the generated profile.

Use in Overweight Individuals

One challenge of developing a non-invasive device which will be placed on the abdomen is the ability to sense the signal of interest in overweight individuals. For those volunteers who reported their weight and height (n=33), we calculated their BMI as outlined in Table 3. From a data analytics perspective, there was no correlation between extracted features and BMI, confirming that it is possible to sense the digital biomarker in all individuals, even those who are overweight. This was establised for two, DWTMean and MaxPower.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1.-24. (canceled)

25. A system to determine status of an endocrine disorder in a subject characterised by abnormal contractility activity of a target pelvic structure, the system comprising:
a sensing module to measure electrical activity of the subject's pelvis at a plurality of spaced-apart time points during the subject's hormonal cycle;
a signal processing module configured to receive electrical activity measurements from the sensing module and isolate from the electrical activity measurements a signal comprising a slow wave contractility frequency representative of the target pelvic structure; and
a processor module operably connected to the signal processing module and configured to:
receive as an input the plurality of signals comprising a slow wave contractility frequency representative of the target pelvic structure;
generate a data profile of the subject comprising the plurality of signals representative of the slow wave contractility frequency of the target pelvic structure in the subject at the plurality of spaced-apart time points during the subject's hormonal cycle;
compare the data profile with a database comprising reference data profiles of reference subjects with or without the endocrine disorder, in which each reference data profile comprises a plurality of signals representative of the slow wave contractility frequency of the target pelvic structure in the reference subject at a plurality of spaced-apart time points during the reference subject's hormonal cycle; and
output the endocrine disorder status of the subject based on the comparison, wherein when the subject is female the hormonal cycle is the menstrual cycle and when the subject is male the hormonal cycle is any 24-hour period.

26. The system according to claim 25, in which the subject's data profile is correlated with endocrine disorder status by employing a classification model generated using reference data profiles from a population of reference subjects with a known endocrine disorder status selected from positive for the disorder, negative for the disorder, risk of developing the disorder, and severity of disorder.

27. The system according to claim 25, in which the signal processing module comprises a filter configured to isolate a signal comprising a slow wave contractility frequency representative of the target pelvic structure.

28. The system according to claim 25, in which the processor module is configured to receive as an additional input a plurality of measurements of at least one non-electrical hormonal cycle parameter taken at a plurality of time points during the subject's hormonal cycle, wherein the generated data profile of the subject comprises the electrical contractility parameter measurements representative of the target pelvic structure and the non-electrical hormonal cycle parameter measurements.

29. The system according to claim 28, in which the subject is female and the non-electrical hormonal cycle parameter is a non-electrical menstrual cycle parameter is selected from pain location, pain intensity, pain type, and bleeding occurrence.

30. The system according to claim 25, in which the endocrine disorder is endometriosis, the subject is female, and the hormonal cycle is the menstrual cycle.

31. The system according to claim 25, to determine whether a non-pregnant female has endometriosis, in which the endocrine disorder is endometriosis, the subject is female, and the hormonal cycle is the menstrual cycle.

32. The system according to claim 30, in which the signal processing module is configured to isolate from each electrical activity measurement a signal comprising a slow wave contractility frequency representative of the uterus and in the range of 0.00 to 0.05 Hz.

33. The system according to claim 30, in which the plurality of spaced apart time points during the subject's menstrual cycle are days 1, 7, 14 and 21, in which the day 14 measurement may be taken at day 14+/−1 day.

34. The system according to claim 25, in which the subject is male and the endocrine disorder is selected from benign prostatic hyperplasia, prostate cancer, and prostatitis.

35. The system according to claim 25, in which the subject is male and the endocrine disorder is selected from benign prostatic hyperplasia, prostate cancer, and prostatitis, and the slow wave contractility frequency representative of the prostate is a contractility frequency of 0.06 to 0.11 Hz.

36. A system to determine whether a non-pregnant female has endometriosis, comprising:
a sensing module to measure electrical activity of the subject's uterus at a plurality of spaced-apart time points during the subject's menstrual cycle, in which the sensing module is a wearable, non-invasive, sensor;
a signal processing module configured to receive electrical activity measurements from the sensing module and isolate from each electrical activity measurement a signal comprising a slow wave contractility frequency representative of the uterus and in the range of 0.00 to 0.05 Hz; and
a processor module operably connected to the signal processing module and configured to:
receive as an input the plurality of signals comprising a slow wave contractility frequency representative of the uterus;
generate a data profile of the subject comprising the plurality of signals representative of the slow wave contractility frequency of the subject's uterus at the plurality of spaced-apart time points during the subject's menstrual cycle;
compare using a classification model the data profile with a database of reference data profiles comprising reference data profiles of reference subjects with or without endometriosis, in which each reference data profile comprises a plurality of signals representative of the slow wave contractility frequency of the reference subject's uterus at a plurality of spaced-apart time points during the reference subject's menstrual cycle;
determine whether the subject has endometriosis based on the comparison; and
provide an output selected from a positive determination of endometriosis and a negative determination of endometriosis.

37. The system according to claim 36, in which the signal processing module comprises a filter configured to isolate a signal comprising a slow wave contractility frequency representative of the uterus and in the range of 0.00 to 0.05 Hz.

38. The system according to claim 36, in which the plurality of time points during the subject's menstrual cycle are days 1, 7, 14 and 21, in which the day 14 measurement may be taken at day 14+/−1 day.

39. A system for treating or preventing endometriosis in a non-pregnant female subject, the system comprising:
a system to determine whether a non-pregnant female has endometriosis according to claim 36; and
a wearable non-invasive uterus stimulating module to apply a stimulation treatment to a uterus to normalise contractility of the uterus,
in which the processor module is configured to:
monitor the subject's menstrual cycle using the signals received from the signal processing module and optionally additional subject data obtained at a plurality of time points during the subject's menstrual cycle; and
transiently actuate the uterus stimulating module during a follicular stage of the subject's menstrual cycle to normalise contractility of the uterus.

40. The system according to claim 39, configured to measure a contractility parameter of the uterus after it has been stimulated, and actuate the uterus stimulating module again if the contractility parameter of the uterus is determined to be abnormal.

41. The system according to claim 25, in which the signal processing module is configured to amplify and digitize the electrical activity measurements.

42. The system according to claim 25, in which the signal processing module comprises a filter configured to isolate the signal comprising a slow wave contractility frequency representative of the subject's uterus.

43. The system according to claim 25, in which the signal processing module is configured to transform the electrical activity measurements into the frequency domain.

44. The system according to claim 25, in which the sensing module is configured to measure an EMG signal of the subjects pelvis.
